SEQUENCE LISTING (3 sequences)

SEQ ID NO: 1 - protein, 195 aa, synthetic construct
  REGION 1..195: note = Description of Artificial Sequence: Synthetic polypeptide
AQQGEEDPQALSIQEGENATMNCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 2 - protein, 500 aa, synthetic construct
  REGION 1..500: note = Description of Artificial Sequence: Synthetic polypeptide
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 3 - protein, 9 aa, Homo sapiens
YLEPGPVTA

(19) United States
(12) Patent Application Publication
(10) Pub. No.: US 2025/0367285 A1
(43) Pub. Date: Dec. 4, 2025

(54) METHODS OF TREATING MELANOMA USING TEBENTAFUSP AND IMMUNE CHECKPOINT INHIBITORS

(71) Applicants: sHAAD ABDULLAH, Abingdon, Oxfordshire (GB); IMMUNOCORE LIMITED, Abingdon, Oxfordshire (GB); ASTRAZENECA AB, Södertälje (SE)

(72) Inventors: Shaad ABDULLAH, Abingdon, Oxfordshire (GB); Christina COUGHLIN, Abingdon, Oxfordshire (GB); Ashok GUPTA, Gaithersburg, MD (US); Shannon MORRIS, Gaithersburg, MD (US)

(21) Appl. No.: 18/871,402

(22) PCT Filed: Jun. 2, 2023
(86) PCT No.: PCT/US2023/067860

§ 371 Date: Dec. 3, 2024

Related U.S. Application Data
(63) Continuation of application No. PCT/US2022/032261, filed on Jun. 3, 2022.

(51) Int. Cl. A61K 39/395; A61K 39/00; A61P 35/00

(52) U.S. Cl. CPC A61K 39/39558; A61P 35/00; A61K 2039/507; A61K 2039/545

(57) ABSTRACT

The present invention relates to the treatment of melanoma in a patient, where the method includes administering to a patient who has or is suspected of having melanoma a therapeutically effective amount of tebentafusp; and a therapeutically effective amount of an immune checkpoint inhibitor. In particular, patients may have metastatic melanoma that is refractory to treatment with an anti-PD(L)1 inhibitor in the metastatic setting or where the patient's melanoma is relapsed following treatment with an anti-PD(L)1 inhibitor.

### 1. CROSS-REFERENCE TO RELATED APPLICATIONS

This application claims the benefit of and priority to International Application No. PCT/US2022/032261, filed Jun. 3, 2022, which is hereby incorporated by reference in its entirety.

### 2. PARTIES TO A JOINT RESEARCH AGREEMENT

The subject matter of this disclosure is subject to a Clinical Study Collaboration Agreement, dated 14 Apr. 2015, between Immunocore Limited and MedImmune Limited.

### 3. SEQUENCE LISTING

The instant application contains a Sequence Listing.

### 4. BACKGROUND OF THE INVENTION

Cutaneous melanoma (CM) arises from the pigmented cells of the skin, melanocytes. CM is a serious form of skin cancer, which currently affects approximately 120,000 new patients per year in the Western World. Just less than half of these cases (estimated at 59,400) occur in Europe with approximately 9,500 new cases of CM diagnosed in the United Kingdom (UK) every year. Moreover, the rate of incidence of the disease is increasing year on year, and cases in the UK have quadrupled since the 1970s. It has been calculated that the lifetime risk of developing malignant CM for women in the UK is 1 in 56 and 1 in 55 for men, and similar results have been observed globally (Rastrelli, 2014).

UM is a rare type of melanoma, with an incidence ranging from 5.3 to 10.9 cases per million (Singh, 2003). Despite its rarity (representing approximately 3% of total melanoma cases, approximately 4,000 cases globally per year), UM is the most frequent primary intraocular malignancy of the adult eye (85%) (Patel, 2011; Maio, 2013). It is an extremely malignant neoplasm that affects the vascular layers of the eye (iris, ciliary body, and choroid) (Maio, 2013). UM is more frequent in men than women, and the majority of cases in the United States (US) occur in the white population (Andreoli, 2015). The cause of UM is not known, but it has been suggested that exposure to ultraviolet rays is the main factor. Known risk factors for UM include fair skin, light-colored eyes, congenital ocular melanocytosis, melanocytoma, and neurofibromatosis.

UM is biologically distinct from CM with differences in the mutational landscape. BRAF and NRAS mutations dominate the landscape in CM, while mutations in guanine nucleotide binding protein (G protein), q polypeptide (GNAQ), and alpha 11 (GNA11) dominate in UM (Shoustari, 2014). In addition, the mode of disease spread is distinct between the 2 disease settings, with hematogenous spread being most common in UM while lymphatic predominates in CM. This leads to the different patterns of metastatic disease with primary liver metastases occurring in UM, contrasted with visceral, bone, and brain metastases that are predominant in CM.

The human glycoprotein 100 (gp100) is one of a panel of melanoma-associated antigens to which the body can mount a natural immune response. The protein is a 661 amino acid melanosomal membrane-associated glycoprotein which is expressed in normal melanocytes and widely overexpressed on the majority of melanoma cancer cells. For example, one study (Trefzer et al., (2006) Melanoma Res. 16(2): 137-45) found that 82% of 192 melanoma metastases from 28 melanoma patients expressed gp100. Several studies reported higher expression levels of gp100 in melanoma tissues (Hofbauer et al., (2004) J Immunother. 27(1): 73-78, Barrow et al., (2006) Clin Cancer Res. 12:764-71). The exact function of the protein is unknown but it appears to be involved in melanosome maturation (Hoashi et al., 2005; Kawakami and Rosenberg, 1997). The gp100 antigen has been, and continues to be, the target of a number of immunotherapy-based melanoma clinical trials (see, e.g., NCT03070392; NCT02570308; and NCT01211262).

The development of tebentafusp (i.e., approved product KIMMTRAK® (tebentafusp-tebn)), a bispecific gp100 peptide HLA-A*02:01 directed T cell receptor—CD3 T cell engager provided a new treatment option for melanoma patients (e.g., metastatic uveal melanoma). The T cell receptor (“TCR”) arm binds to a gp100 peptide presented by human leukocyte antigen-A*-2:01 (HLA A*02:01) on the cell surface of melanoma tumor cells. The mechanism of action of such a therapeutic is significantly different to other immunotherapy predecessors and results in the rapid and potent redirection of non-gp100 specific T cells to kill gp100 positive cells in vitro; thus there is a sound rationale for expecting both improved clinical efficacy results in patients and on-target, off-tumor toxicities from activity against gp100 positive normal tissues such as skin melanocytes.

Although checkpoint inhibition has delivered remarkable results in metastatic melanoma, there remains a significant patient population with disease progression early after initiating therapy (Robert, 2015; Larkin, 2015; Wolchok, 2013). Resistance mechanisms to checkpoint inhibition is under study to identify potential combinations that might revert checkpoint refractory disease. In metastatic melanoma, emerging biology suggests that checkpoint inhibitor resistance may align with tumors that have largely been ignored by the immune system (Tumeh, 2014). Optimal responses to programmed death-1 (PD-1)-directed therapy require the presence of CD8+ T cells in the tumor microenvironment; therefore, new therapeutic approaches and combinations that recruit these effectors to immunologically “silent” tumors may overcome pre-existing resistance to checkpoint blockade (Chen, 2013; Tumeh, 2014).

Therefore, even with the development of tebentafusp and immune checkpoint inhibitors, there remains a need to improve overall survival of melanoma patients, in particular, those patients whose melanoma is refractory or resistant to previous treatment with an immune checkpoint inhibitor.

### 5. SUMMARY OF THE INVENTION

Provided herein is a method of treating a melanoma in a patient, where the method includes administering to a patient, who has or is suspected of having melanoma, a therapeutically effective amount of tebentafusp; and a therapeutically effective amount of an immune checkpoint inhibitor. The methods provided herein are based, in part, on recent biologic evidence that indicates that optimal responses to programmed cell death-1 (PD-1) directed therapy require the presence of CD8⁺ T cells in the tumor microenvironment and thus therapies such as tebentafusp (IMCgp100) that recruit these effector cells to the tumor may overcome pre-existing resistance to checkpoint blockade. A previous in vitro study showed the combination of tebentafusp with an anti-PD1 antibody increased tebentafusp efficacy (see Petrovic et al., ImmTAC redirect exhausted tumor-infiltrating T-cells: An effect enhanced by pembrolizumab against PD-L1+ tumors, Ann. Oncol., 32, S856 (2021)). This report was limited to pembrolizumab but it suggests the combination of tebentafusp (IMCgp100) with immune checkpoint inhibition may have enhanced activity in patients with pre-existing resistance to an immune checkpoint inhibitor.

In one aspect, the present disclosure provides a method of treating melanoma in a patient, comprising: administering to a patient who has or is suspected of having melanoma: a therapeutically effective amount of tebentafusp; and a therapeutically effective amount of a first immune checkpoint inhibitor.

In another aspect, the present disclosure provides a method of treating melanoma in a patient, comprising: administering to a patient who is refractory to or relapsed following a prior checkpoint inhibitor treatment: a therapeutically effective amount of tebentafusp; and a therapeutically effective amount of a first immune checkpoint inhibitor. In some embodiments, the prior immune checkpoint inhibitor treatment comprises an inhibitor of PD(L)1, CTLA-4, or a combination thereof.

In some embodiments, the method further comprises administering a second immune checkpoint inhibitor.

In some embodiments, the first immune checkpoint inhibitor or the second immune checkpoint inhibitor is an inhibitor of PD(L)1. In some embodiments, the inhibitor is a monoclonal antibody that binds PD(L)1. In some embodiments, the monoclonal antibody that binds PD(L)1 is selected from: durvalumab, atezolizumab, BMS-936559, avelumab, pembrolizumab, nivolumab, dostarlimab, and cemiplimap. In some embodiments, the monoclonal antibody that bind PD(L)1 is durvalumab.

In some embodiments, the first immune checkpoint inhibitor and/or the second immune checkpoint inhibitor is an inhibitor of CTLA-4. In some embodiments, the inhibitor is a monoclonal antibody that binds CTLA-4. In some embodiments, the monoclonal antibody that binds CTLA-4 is selected from: tremelimumab, ipilimumab, quavonlimab, zalifrelimab, GIGA-564, CBT-509, and AGEN1181. In some embodiments, the monoclonal antibody that binds CTLA-4 is tremelimumab.

In some embodiments, the melanoma is cutaneous melanoma. In some embodiments, the cutaneous melanoma is metastatic cutaneous melanoma. In some embodiments, the melanoma is refractory or resistant. The method of claim 15, wherein the patient was previously treated with an inhibitor of PD(L)1.

In some embodiments, the patient has a BRAF mutation. In some embodiments, the patient was previously treated with an approved BRAF-based therapy.

In some embodiments, the patient is HLA-A2-positive.

In some embodiments, the tebentafusp and the first immune checkpoint inhibitor are administered separately.

In some embodiments, the tebentafusp is administered weekly.

In some embodiments, the first immune checkpoint inhibitor is administered once every four weeks.

In some embodiments, the second immune checkpoint inhibitor is administered once every four weeks.

In some embodiments, the first immune checkpoint inhibitor and the second immune checkpoint inhibitor are administered concurrently.

In some embodiments, the first immune checkpoint inhibitor and the second immune checkpoint inhibitor are administered separately.

In some embodiments, the first immune checkpoint inhibitor, the second immune checkpoint inhibitor, or both, are first administered about 15 days after the first administration of the tebentafusp.

In some embodiments, the tebentafusp is administered at a dose ranging from about 10 mcg to about 68 mcg.

In some embodiments, the tebentafusp is administered at a dose ranging from about 10 mcg to about 50 mcg.

In some embodiments, the tebentafusp is administered at a dose ranging from about 68 mcg to about 200 mcg.

In some embodiments, the first immune checkpoint inhibitor, the second immune checkpoint inhibitor, or both, are administered at a dose ranging from about 1 mg/kg to about 20 mg/kg.

In some embodiments, the first immune checkpoint inhibitor, the second immune checkpoint inhibitor, or both, are administered at a dose ranging from about 0.5 mg/kg to about 10 mg/kg.

In some embodiments, the first immune checkpoint inhibitor, the second immune checkpoint inhibitor, or both, are administered at a dose ranging from about 0.5 mg/kg to about 1 mg/kg.

In some embodiments, the first immune checkpoint inhibitor is durvalumab. In some embodiments, the tebentafusp is administered at a dose ranging from about 10 mcg to about 68 mcg and the durvalumab is administered at a dose ranging from about 1 mg/kg to about 20 mg/kg. In some embodiments, the tebentafusp is administered about once weekly. In some embodiments, the durvalumab is administered about once every four weeks. In some embodiments, the durvalumab is first administered about 15 days after the first administration of the tebentafusp.

In some embodiments, the first immune checkpoint inhibitor is tremelimumab. In some embodiments, the tebentafusp is administered at a dose ranging from about 10 mcg to about 50 mcg and the tremelimumab is administered at a dose ranging from about 0.5 mg/kg to about 10 mg/kg. In some embodiments, the tebentafusp is administered about once weekly. In some embodiments, the tremelimumab is administered about once every four weeks. In some embodiments, the tremelimumab is first administered about 15 days after the first administration of the tebentafusp.

In some embodiments, the first immune checkpoint inhibitor is durvalumab and the second immune checkpoint is tremelimumab. In some embodiments, the tebentafusp is administered at a dose ranging from about 10 mcg to about 50 mcg, the durvalumab is administered at a dose ranging from about 1 mg/kg to about 20 mg/kg, and the tremelimumab is administered at a dose ranging from about 0.5 mg/kg to about 1 mg/kg. In some embodiments, the tebentafusp is administered about once weekly. In some embodiments, the durvalumab is administered about once every four weeks. In some embodiments, the durvalumab is first administered about 15 days after the first administration of the tebentafusp. In some embodiments, the tremelimumab is administered about once every four weeks. In some embodiments, the tremelimumab is first administered about 15 days after the first administration of the tebentafusp. In some embodiments, the first immune checkpoint inhibitor and the second immune checkpoint inhibitor are administered concurrently. In some embodiments, the first immune checkpoint inhibitor and the second immune checkpoint inhibitor are administered separately.

In some embodiments, tebentafusp is administered via intravenous infusion, subcutaneous infusion, intramuscular infusion, enteral administration, inhalation, or intranasal.

In some embodiments, the first immune checkpoint inhibitor, the second immune checkpoint inhibitor, or both are administered via intravenous infusion, subcutaneous infusion, intramuscular infusion, enteral administration, inhalation, or intranasal.

In another aspect, the present disclosure provides a method of treating metastatic cutaneous melanoma in a patient, comprising: administering: tebentafusp at a dose ranging from about 10 mcg to about 68 mcg; and a PD(L)1 inhibitor at a therapeutically effective amount, wherein the tebentafusp is administered about once weekly, wherein the PD(L)1 inhibitor is administered about once every four weeks, and wherein the PD(L)1 inhibitor is first administered about 15 days after the first administration of the tebentafusp. In some embodiments, the PD(L)1 inhibitor is a monoclonal antibody that binds PD(L)1. In some embodiments, the monoclonal antibody that binds PD(L)1 is selected from: durvalumab, atezolizumab, BMS-936559, and Avelumab. In some embodiments, the PD(L)1 inhibitor is durvalumab.

In another aspect, the present disclosure provides a method of treating metastatic cutaneous melanoma in a patient, comprising: administering: tebentafusp at a dose ranging from about 10 mcg to about 68 mcg; and durvalumab at a dose ranging from about 1 mg/kg to about 20 mg/kg, wherein the tebentafusp is administered about once weekly, wherein the durvalumab is administered about once every four weeks, and wherein the durvalumab is first administered about 15 days after the first administration of the tebentafusp.

In another aspect, the present disclosure provides a method of treating metastatic cutaneous melanoma in a patient, comprising: administering: tebentafusp at a dose ranging from about 10 mcg to about 50 mcg; and a CTLA-4 inhibitor at a therapeutically effective amount, wherein the tebentafusp is administered about once weekly, wherein the CTLA-4 inhibitor is administered about once every four weeks, and wherein the CTLA-4 inhibitor is first administered about 15 days after the first administration of the tebentafusp.

In some embodiments, the CTLA-4 inhibitor is a monoclonal antibody that binds CTLA-4.

In some embodiments, the monoclonal antibody that binds CTLA-4 is selected from: tremelimumab, ipilimumab, quavonlimab, zalifrelimab, GIGA-564, CBT-509, and AGEN1181.

In some embodiments, the CTLA-4 inhibitor is tremelimumab.

In another aspect, the present disclosure provides a method of treating metastatic cutaneous melanoma in a patient, comprising: administering: tebentafusp at a dose ranging from about 10 mcg to about 50 mcg; and tremelimumab at a dose ranging from about 0.5 mg/kg to about 10 mg/kg, wherein the tebentafusp is administered about once weekly, wherein the tremelimumab is administered about once every four weeks, and wherein the tremelimumab is first administered about 15 days after the first administration of the tebentafusp.

In another aspect, the present disclosure provides a method of treating metastatic cutaneous melanoma in a patient, comprising: administering: tebentafusp at a dose ranging from about 10 mcg to about 50 mcg; a PD(L)1 inhibitor at a therapeutically effective amount; and a CTLA-4 inhibitor at a therapeutically effective amount, wherein the tebentafusp is administered about once weekly, wherein the PD(L)1 inhibitor is administered about once every four weeks, wherein the CTLA-4 inhibitor is administered about once every four weeks, and wherein the PD(L)1 inhibitor and the CTLA-4 inhibitor are first administered about 15 days after the first administration of the tebentafusp.

In some embodiments, the inhibitor is a monoclonal antibody that binds PD(L)1.

In some embodiments, the monoclonal antibody that binds PD(L)1 is selected from: durvalumab, atezolizumab, BMS-936559, and Avelumab.

In some embodiments, the PD(L)1 inhibitor is durvalumab.

In some embodiments, the inhibitor is a monoclonal antibody that binds CTLA-4.

In some embodiments, the monoclonal antibody that binds CTLA-4 is selected from: tremelimumab, ipilimumab, quavonlimab, zalifrelimab, GIGA-564, CBT-509, and AGEN1181.

In some embodiments, the CTLA-4 inhibitor is tremelimumab.

In another aspect, the present disclosure provides is a method of treating metastatic cutaneous melanoma in a patient, comprising: administering: tebentafusp at a dose ranging from about 10 mcg to about 68 mcg; durvalumab at a dose ranging from about 1 mg/kg to about 20 mg/kg; and tremelimumab at a dose ranging from about 0.5 mg/kg to about 10 mg/kg, wherein the tebentafusp is administered about once weekly, wherein the durvalumab is administered about once every four weeks, wherein the tremelimumab is administered about once every four weeks, and wherein the durvalumab and the tremelimumab are first administered about 15 days after the first administration of the tebentafusp.

### 6. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A is a schematic of the study design for the clinical trial described in Example 1.

FIG. 1B is a schematic showing dose schedules for the clinical trial (arms 1-3 and arm 4) described in Example 1. First top panel shows dose schedule for treatment period (cycles 1-13) for arms 1-3. First bottom panel shows dose schedule for maintenance period (cycles 14-25) for arms-3. Second top panel shows dose schedule for treatment period for arm 4a and second bottom panel shows dose schedule for treatment period for arm 4b.

FIG. 1C is a schematic showing schedules for the clinical trial (arms-1-3 and arm 4) described in Example 1.

FIG. 2 provides a table showing the baseline demographics for the clinical trial described in Example 2.

FIG. 3 provides a table showing the treatment-related adverse events for the clinical trial described in Example 2.

FIG. 4 shows a bar chart of change from baseline (%) in tumor size of patients who received prior anti-PD(L)1 therapy. Patients ordered based on baseline (%) and colored according to study Arm. * indicates patients evaluable for response (N=91); tumor shrinkage at any time prior to subsequent therapy. + indicates patients with >12 month survival.

FIG. 5 shows a plot illustrating probability of shrinkage (durability) (y-axis) versus duration of shrinkage (months) (x-axis) in patients who received prior anti-PD(L) therapy. Patients are grouped by arm of study. * indicates exclusion of 2 patients on a 3× per week regimen of tebentafusp as this was considered impractical for long-term treatment.

FIG. 6 shows a plot illustrating change from baseline (%) in tumor size (y-axis) from time from first dose (months) (x-axis). Patients are identified according to status: refractory, resistant, or new lesion. Data was limited to patients with known best overall response to prior anti-PD(L)1 therapy. Tumor shrinkage is indicated regardless of whether new lesions were identified. Resistant=best response CR/PR/SD to prior PD(L)1. Refractory=best response of PD to prior anti-PD(L)1. Complete response (CR), a partial response (PR), progressive disease (PD), and stable disease (SD).

FIG. 7 shows survival probability (y-axis) over a 42-month time frame with patients grouped according to status of prior anti-PD(L) therapy: refractory or resistance. + indicates censored. Data was limited to patients with known best overall response to prior anti-PD(L)1 therapy (N=96). Resistant=best response CR/PR/SD to prior PD(L)1. Refractory=best response of PD to prior anti-PD(L)1 therapy. Complete response (CR), a partial response (PR), progressive disease (PD), and stable disease (SD).

FIG. 8 shows survival probability (y-axis) for patients in study Arm 1 (tebentafusp and durvalumab) grouped according to status of prior anti-PD(L)1 therapy: refractory or resistance. + indicates censored. * indicates no durvalumab (durva) and <10 mg/kg durva groups include Arm 4 monotherapy patients who have limited follow-up. † indicates durva does not include 2 patients who were assigned to receive durvalumab but only received tebentafusp monotherapy.

FIG. 9 illustrates association between tumor shrinkage and overall survival (OS) for patients in Arm 1 (tebentafusp and durvalumab). Top panel shows overall survival with treatment schedules having exceeded 12 months in purple and treatment schedules of less than or equal to 12 months in gray. Each bar indicates OS for a single patient. + indicates censored. Bottom panel shows change from baseline (%) for tumor measurements corresponding to the same patients immediately above in the top panel. Tumor size was measured as sum of longest diameters or short axis of target lesions according to RECIST 1.1 (per investigator). Best % change in target lesion size is maximum % reduction from baseline or minimum % increase from baseline (in absence of reduction). 52 of 58 patients had at least one evaluable post-baseline scan. Complete response (CR), a partial response (PR), progressive disease (PD), and stable disease (SD).

FIG. 10 shows similar associations with overall survival ability (OS) between metastatic uveal melanoma (mUM) and metastatic cutaneous melanoma (mCM). * indicates 3% and 6% of patients in Study 201 and Study 202, respectively, had no change in tumor size. April 2022 data cut off for survival data. Tumor shrinkage and increase for IMCgp100-202 (N=230).

FIG. 11 shows survival probability comparing Arm 1 (tebentafusp and durvalumab) and Arm 2 (tebentafusp, durvalumab and tremelimumab) over 66 month time course.

### 7. DETAILED DESCRIPTION OF THE INVENTION

### 7.1. Definitions

As used herein, the term “antibody” refers to immunoglobulin molecules and immunologically active portions of immunoglobulin molecules, i.e., molecules that contain an antigen binding site that specifically binds an antigen, whether natural or partly or wholly synthetically produced. The term “antibody” includes antibody fragments, derivatives, functional equivalents and homologues of antibodies, humanized antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. A humanized antibody may be a modified antibody having the variable regions of a non-human, e.g. murine, antibody and the constant region of a human antibody. Methods for making humanized antibodies are described in, for example, U.S. Pat. No. 5,225,539. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to herein as “mAb”.

As used herein, the term “baseline” refers to measurements taken at the time of first administration of tebentafusp or measurements taken just prior to administration of tebentafusp.

As used herein, the term “binding affinity” (inversely proportional to the equilibrium constant KD) and “binding half-life” (expressed as T1/2) can be determined by any appropriate method. It will be appreciated that doubling the affinity of a TCR results in halving the KD. T1/2 is calculated as ln2 divided by the off-rate (koff). So doubling of T1/2 results in a halving in koff KD and koff values for TCRs are usually measured for soluble forms of the TCR, i.e. those forms which are truncated to remove hydrophobic transmembrane domain residues. Preferably the binding affinity or binding half-life of a given TCR is measured several times, for example 3 or more times, using the same assay protocol, and an average of the results is taken. In a preferred embodiment these measurements are made using the Surface Plasmon Resonance (BIAcore) method of Example 3 of WO2011/001152.

As used herein, the term “censored” refers to statistical analysis with respect to overall survival. For example, any patient not known to have died at the time of analysis will be censored based on the last recorded date on which the patient was known to be alive.

Throughout this specification and claims, the word “comprise” or variations such as “comprises” or “comprising”, will be understood to imply the inclusion of a stated integer or group of integers but not the exclusion of any other integer or group of integers.

As used herein, the term “RECIST” refers to a standard way to measure how well a cancer patient responds to treatment. It is based on whether tumors shrink, stay the same, or get bigger. To use RECIST, there must be at least one tumor that can be measured on x-rays, CT scans, or MRI scans. The types of response a patient can have include: a complete response (CR), a partial response (PR), progressive disease (PD), and stable disease (SD). Also called Response Evaluation Criteria In Solid Tumors.

As used herein, the term “relapse” or “relapsed” refers to the return of a disease (e.g., cancer) of the signs and symptoms of a disease after a period of improvement.

As used herein, the term “refractory” refers to a disease (e.g., cancer) that does not respond to treatment.

As used herein, the term “T cell receptor” or “TCR” are described using the International Immunogenetics (IMGT) TCR nomenclature, and links to the IMGT public database of TCR sequences. The unique sequences defined by the IMGT nomenclature are widely known and accessible to those working in the TCR field. For example, they can be found in the “T cell Receptor Factsbook”, (2001) LeFranc and LeFranc, Academic Press, ISBN 0-12-441352-8; Lefranc, (2011), Cold Spring Harb Protoc 2011(6): 595-603; Lefranc, (2001), Curr Protoc Immunol Appendix 1: Appendix 1O; Lefranc, (2003), Leukemia 17(1): 260-266, and on the IMGT website (www.IMGT.org).

As used herein, the term “tebentafusp” refers to the approved product KIMMTRAK® (tebentafusp-tebn), a bispecific gp100 peptide HLA-A*02:01 directed T cell receptor—CD3 T cell engager. The T cell receptor (“TCR”) arm binds to a gp100 peptide presented by human leukocyte antigen-A*-2:01 (HLA A*02:01) on the cell surface of melanoma cells.

As used herein, the term “therapeutically effective” refers to the amount of a compound (e.g., antibody) that, when administered to a patient for treating a disease (e.g., melanoma), is sufficient to effect treatment or reduce symptoms associated with the disease. Here, effecting treatment includes, without limitation, increasing overall survival rate at 1 year or at 2 years, improving the RECIST response rate, and/or shrinking a tumor.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

It must be noted that as used herein and in the appended claims, the singular forms “a,” “an,” and “the” include plural referents unless the context clearly dictates otherwise. Thus, for example, reference to “an antibody or antigen binding fragment” includes a plurality of such antibodies and antigen binding fragments and reference to “the recombinant adeno-associated virus” includes reference to one or more recombinant adeno-associated viruses and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### 7.1. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### 7.2. Method of Treating Melanoma

Provided herein are methods of treating melanoma in a patient, where the method includes: administering to a patient who has or is suspected of having melanoma: a therapeutically effective amount of tebentafusp; and a therapeutically effective amount of an immune checkpoint inhibitor. In some embodiments, the method also includes administering to the patient a second immune checkpoint inhibitor.

Provided herein are methods of treating melanoma in a patient, where the method includes: administering to a patient who is refractory to or relapsed following a prior checkpoint inhibitor treatment: a therapeutically effective amount of tebentafusp; and a therapeutically effective amount of a first immune checkpoint inhibitor. In some embodiments where the patients received treatment with a prior immune checkpoint inhibitor, the inhibitor was an inhibitor of PD(L)1, CTLA-4, or a combination thereof.

Provided herein are methods of treating metastatic cutaneous melanoma in a patient, where the method includes: administering: tebentafusp at a dose ranging from about 10 mcg to about 68 mcg; and a PD(L)1 inhibitor at a therapeutically effective amount, wherein the tebentafusp is administered about once weekly, wherein the PD(L)1 inhibitor is administered about once every four weeks, and wherein the PD(L)1 inhibitor is first administered about 15 days after the first administration of the tebentafusp. In some embodiments, the PD(L)1 inhibitor is any of the PD(L)1 inhibitors described herein (see Section 6.4.1). In some embodiments, the PD(L)1 inhibitor is durvalumab.

Provided herein is a method of treating metastatic cutaneous melanoma in a patient, where the method includes: administering: tebentafusp at a dose ranging from about 10 mcg to about 68 mcg; and durvalumab at a dose ranging from about 1 mg/kg to about 20 mg/kg, wherein the tebentafusp is administered about once weekly, wherein the durvalumab is administered about once every four weeks, and wherein the durvalumab is first administered about 15 days after the first administration of the tebentafusp.

Provided herein are methods of treating metastatic cutaneous melanoma in a patient, where the method includes: administering: tebentafusp at a dose ranging from about 10 mcg to about 50 mcg; and a CTLA-4 inhibitor at a therapeutically effective amount, wherein the tebentafusp is administered about once weekly, wherein the CTLA-4 inhibitor is administered about once every four weeks, and wherein the CTLA-4 inhibitor is first administered about 15 days after the first administration of the tebentafusp. In some embodiments, the CTLA-4 inhibitor is any of the CTLA-4 inhibitors described herein (see Section 6.4.2). In some embodiments, the CTLA-4 inhibitor is tremelimumab.

Provided herein are methods of treating metastatic cutaneous melanoma in a patient, where the method includes: administering: tebentafusp at a dose ranging from about 10 mcg to about 68 mcg; and tremelimumab at a dose ranging from about 0.5 mg/kg to about 10 mg/kg, wherein the tebentafusp is administered about once weekly, wherein the tremelimumab is administered about once every four weeks, and wherein the tremelimumab is first administered about 15 days after the first administration of the tebentafusp.

Provided herein are methods of treating metastatic cutaneous melanoma in a patient, where the method includes: administering: tebentafusp at a dose ranging from about 10 mcg to about 50 mcg; a PD(L)1 inhibitor at a therapeutically effective amount; and a CTLA-4 inhibitor at a therapeutically effective amount, wherein the tebentafusp is administered about once weekly, wherein the PD(L)1 inhibitor is administered about once every four weeks, wherein the CTLA-4 inhibitor is administered about once every four weeks, and wherein the PD(L)1 inhibitor and the CTLA-4 inhibitor are first administered about 15 days after the first administration of the tebentafusp. In some embodiments, the PD(L)1 inhibitor is any of the PD(L)1 inhibitors described herein (see Section 6.4.1). In some embodiments, the PD(L)1 inhibitor is durvalumab. In some embodiments, the CTLA-4 inhibitor is any of the CTLA-4 inhibitors described herein (see Section 6.4.2). In some embodiments, the CTLA-4 inhibitor is tremelimumab.

Provided herein are methods of treating metastatic cutaneous melanoma in a patient, comprising: administering: tebentafusp at a dose ranging from about 10 mcg to about 68 mcg; durvalumab at a dose ranging from about 1 mg/kg to about 20 mg/kg; and tremelimumab at a dose ranging from about 0.5 mg/kg to about 10 mg/kg, wherein the tebentafusp is administered about once weekly, wherein the durvalumab is administered about once every four weeks, wherein the tremelimumab is administered about once every four weeks, and wherein the durvalumab and the tremelimumab are first administered about 15 days after the first administration of the tebentafusp.

Provided herein are methods of treating metastatic cutaneous melanoma in patient where the method includes administering, to a patient who has or is suspected of having metastatic cutaneous melanoma, where the method includes: administering: tebentafusp at a dose ranging from about 68 mcg to about 200 mcg, wherein the tebentafusp is administered about once weekly.

After administration of tebentafusp and the first immune checkpoint inhibitor, the second immune checkpoint inhibitor, or a combination thereof, the percent change from baseline in tumor size ranges from about 18% to about 44% (e.g., any of the values of subranges therein).

After administration of tebentafusp and the first immune checkpoint inhibitor, the second immune checkpoint inhibitor, or a combination thereof, the percent change from baseline in tumor size of at least 18% (e.g., at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43% or at least 44%).

After administration of tebentafusp and the first immune checkpoint inhibitor, the second immune checkpoint inhibitor, or a combination thereof, the patient has a RECIST response rate ranging from about 5% to about 15% (e.g., any of the values of subranges therein).

After administration of tebentafusp and the first immune checkpoint inhibitor, the second immune checkpoint inhibitor, or a combination thereof, the patient has a RECIST response rate of at least 5% (e.g., at least 6%, at least 7%, at least 8%, at least 9%, at least 10%, at least 11%, at least 12%, at least 13%, at least 14%, or at least 15%). In some embodiments, the RECIST response rate is relative to timepoint just prior to initiation of treatment.

After administration of tebentafusp and the first immune checkpoint inhibitor, the second immune checkpoint inhibitor, or a combination thereof, the overall survival (OS) rate at 1 year is about 73 to about 78% (e.g., 73% to about 77%, about 73% to about 76%, about 73% to about 75%, about 73% to about 74%, about 74% to about 78%, about 74% to about 77%, about 74% to about 76%, about 74% to about 75%, about 75% to about 78%, about 75% to about 77%, about 75% to about 76%, about 76% to about 78%, about 76% to about 77%, or about 77% to about 78%).

After administration of tebentafusp and the first immune checkpoint inhibitor, the second immune checkpoint inhibitor, or a combination thereof, the overall survival (OS) rate at 1 year is at least 73% (e.g., at least 74%, at least 75%, at least 76%, at least 77%, or at least 78%). In some embodiments, wherein, after administration of tebentafusp and the first immune checkpoint inhibitor, the second immune checkpoint inhibitor, or a combination thereof, the overall survival (OS) rate at 1 year is greater than about 50%, about 55%, about 60%, about 65%, about 70%, about 75%, about 80%, about 85%, about 90%, about 95%, or about 99%.

### 7.3. Tebentafusp

In some embodiments, tebentafusp is the approved product KIMMTRAK® (tebentafusp-tebn), a bispecific gp100 peptide HLA-A*02:01 directed T cell receptor—CD3 T cell engager. The T cell receptor (“TCR”) arm binds to a gp100 peptide presented by human leukocyte antigen-A*-2:01 (HLA A*02:01) on the cell surface of uveal melanoma tumor cells. The anti-CD3 T cell engaging arm activates polyclonal T cells to release inflammatory cytokines and cytolytic proteins, which results in direct lysis of melanoma tumor cells. KIMMTRAK® is indicated for the treatment of HLA A*02:01-positive adult patients with unresectable or metastatic melanoma. See Prescribing Information, Exhibit F, sections 11 and 12.

Tebentafusp is composed of an alpha chain and a beta chain. The alpha chain comprises a TCR alpha chain variable region domain. The beta chain comprises a TCR beta chain variable region domain and a single-chain variable fragment (“scFv”) anti-CD3 antibody. The two chains are covalently bonded via a disulfide bond between the cysteine at position 157 on the alpha chain (α157) and the cysteine in position 427 on the beta chain (β427) (indicated with asterisks, *, in the amino acid sequences below).

The amino acid sequence of tebentafusp is provided below. The alpha chain is composed of 195 amino acid residues and the beta chain is composed of 500 amino acid residues. The linker sequence between the two variable domains of the scFv antibody (24 amino acids) and the linker sequence between the scFv and TCR β chain variable region (5 amino acids) are underlined. The initiator methionine residues are post-translationally removed from both alpha and beta chains.

Amino acid sequence of tebentafusp-tebn:

Alpha chain
(SEQ ID NO: 1)
AQQGEEDPQALSIQEGENATMNCSYKTSINNLQWYRQNSGRGLVH
LILIRSNEREKHSGRLRVTLDTSKKSSSLLITASRAADTASYFCA
TDGSTPMQFGKGTRLSVIANIQKPDPAVYQLRDSKSSDKSVCLFT
DFDSQTNVSQSKDSDVYITDKC*VLDMRSMDFKSNSAVAWSNKSD
FACANAFNNSIIPEDT
Beta chain
(SEQ ID NO: 2)
AIQMTQSPSSLSASVGDRVTITCRASQDIRNYLNWYQQKPGKAPK
LLIYYTSRLESGVPSRFSGSGSGTDYTLTISSLQPEDFATYYCQQ
GNTLPWTFGQGTKVEIKGGGGSGGGGSGGGGSGGGGSGGGSEVQL
VESGGGLVQPGGSLRLSCAASGYSFTGYTMNWVRQAPGKGLEWVA
LINPYKGVSTYNQKFKDRFTISVDKSKNTAYLQMNSLRAEDTAVY
YCARSGYYGDSDWYFDVWGQGTLVTVSSGGGGSDGGITQSPKYLF
RKEGQNVTLSCEQNLNHDAMYWYRQDPGQGLRLIYYSWAQGDFQK
GDIAEGYSVSREKKESFPLTVTSAQKNPTAFYLCASSWGAPYEQY
FGPGTRLTVTEDLKNVFPPEVAVFEPSEAEISHTQKATLVCLATG
FYPDHVELSWWVNGKEVHSGVC*TDPQPLKEQPALNDSRYALSSR
LRVSATFWQDPRNHFRCQVQFYGLSENDEWTQDRAKPVTQIVSAE
AWGRAD

### 7.4. Immune Checkpoint Inhibitors

In some embodiments, the methods described herein include an immune checkpoint inhibitor (e.g., a first immune checkpoint inhibitor and a second immune checkpoint inhibitor).

In some embodiments, an immune checkpoint inhibitor comprises an agent that blocks either directly or indirectly signaling associated with CTLA-4, PD-1, PD-L1, PD-L2, LAG-3, TIM-3, TIGIT, or a combination thereof.

In some embodiments, the immune-checkpoint inhibitor is a natural or engineered ligand of an inhibitory immune checkpoint molecule, including, for example, ligands of CTLA-4 (e.g., B7.1, B7.2), ligands of TIM3 (e.g., Galectin-9), ligands of A2a Receptor (e.g., adenosine, Regadenoson), ligands of LAG3 (e.g., MHC class I or MHC class II molecules), ligands of BTLA (e.g., HVEM, B7-H4), ligands of KIR (e.g., MHC class I or MHC class II molecules), ligands of PD-1 (e.g., PD-L1, PD-L2), ligands of IDO (e.g., NKTR-218, Indoximod, NLG919), and ligands of CD47 (e.g., SIRP-alpha receptor). In some embodiments, the immune checkpoint inhibitor is an antibody that targets an inhibitory immune checkpoint protein. In some embodiments, the immune checkpoint inhibitor is an antibody selected from the group consisting of anti-CTLA-4 (e.g., Ipilimumab, Tremelimumab, KAHR-102), anti-TIM3 (e.g., F38-2E2, ENUM005), anti-LAG3 (e.g., BMS-986016, IMP701, IMP321, C9B7W), anti-KIR (e.g., Lirilumab and IPH2101), anti-PD-1 (e.g., Nivolumab, Pidilizumab, Pembrolizumab, BMS-936559, atezolizumab, Lambrolizumab, MK-3475, AMP-224, AMP-514, STI-A1 1 10, TSR-042), anti-PD-L1 (e.g., KY-1003 (EP20120194977), MCLA-145, RG7446, BMS-936559, MEDI-4736, MSB0010718C, AUR-012, STI-AI OIO, PCT/US2001/020964, MPDL3280A, AMP-224, Dapirolizumab pegol (CDP-7657), MEDI-4920), anti-CD73 (e.g., AR-42 (OSU-HDAC42, HDAC-42, AR42, AR42, OSU-HDAC-42, OSU-HDAC-42, NSC D736012, HDAC-42, HDAC42, HDAC42, NSCD736012, NSC-D736012), MEDI-9447), anti-B7-H3 (e.g., MGA271, DS-5573a, 8H9), anti-CD47 (e.g., CC-90002, TTI-621, VLST-007), anti-BTLA, anti-VIST A, anti-A2aR, anti-B7-1, anti-B7-H4, anti-CD52 (such as alemtuzumab), anti-IL-10, anti-IL-35, and anti-TGF-β (such as Fresolumimab).

In some embodiments, the antibody is a monoclonal antibody. In some embodiments, the antibody is an antigen-binding fragment selected from the group consisting of Fab, Fab′, F(ab′)2, Fv, scFv, and other antigen-binding subsequences of the full length antibody. In some embodiments, the antibody is a human, humanized, or chimeric antibody. In some embodiments, the antibody is a bispecific antibody, a multi-specific antibody, a single domain antibody, a fusion protein comprising an antibody portion, or any other functional variants or derivatives thereof.

### 7.4.1. PD(L)1 Inhibitor

In some embodiments, an immune checkpoint inhibitor (e.g., a first immune checkpoint inhibitor and a second immune checkpoint inhibitor) is an inhibitor of PD(L)1. As used herein, “PD(L)1” refers to both PD-L1 and PD-1. As used herein, “PD-L1” refers to programmed cell death-ligand 1 (PD-L1), which is also known as cluster of differentiation 274 (CD274) or B7 homolog 1 (B7-H1). PD-L1 is expressed by hematopoietic and non-hematopoietic cells, such as T cells and B cells. In particular, interaction of PD-L1 with its receptor inhibits T-cell activation and cytokine production. As used herein, “PD-1” refers to programmed cell death protein 1 (PDCD1). PD-1 is an immune-inhibitory receptor expressed in activated T cells; it is involved in the regulation of T-cell functions, including those of effector CD8+ T cells.

In some embodiments, an inhibitor of PD-L1 includes an inhibitor that blocks the interaction between PD-L1 and PD-1. Without wishing to be bound by theory, when a PD-1 molecule expressed on or at the surface a cell (e.g., a T cell) interacts with the inhibitory checkpoint molecule PD-L1, this transmit an inhibitory signal thereby blocking the T cell activation.

In some embodiments, an inhibitor of PD-L1 is a monoclonal antibody that binds to PD-L1. This binding blocks the interaction with PD-1 or inhibits and downstream signaling that results from the interaction between PD-1 and PD-L1. Non-limiting examples of monoclonal antibodies that bind PD-L1 include are described herein, including, durvalumab, atezolizumab, BMS-936559, and Avelumab.

Any of the known anti-PD(L)1 antibodies provided in the following publications can be used: U.S. Pat. Nos. 7,943,743, 7,722,868, 8,217,149, 8,383,796, 8,552,154, and 9,102,725; and U.S. Patent Application Publication Nos. US20140341917, and US20150203580; and International Patent Application No, PCT/US2001/020964, each of which is herein incorporated by reference in its entirety. For example, anti-PD(L)1 antibodies include: Nivolumab, Pidilizumab, Pembrolizumab, BMS-936559, atezolizumab, Lambrolizumab, MK-3475, AMP-224, AMP-514, STI-A1 1 10, TSR-042), anti-PD-L1 (e.g., KY-1003 (EP20120194977), MCLA-145, RG7446, BMS-936559, MEDI-4736, MSB0010718C, AUR-012, STI-AI OIO, PCT/US2001/020964, MPDL3280A, AMP-224, Dapirolizumab pegol (CDP-7657) and MEDI-4920.

In some embodiments, the monoclonal antibody that bind to PD-L1 is durvalumab. Durvalumab is a human immunoglobulin G (IgG) 1 kappa monoclonal antibody (mAb) directed against human PD-L1. Durvalumab is expressed in Chinese hamster ovary cells and has an overall molecular weight of approximately 149 kDa. Durvalumab selectively binds human PD-L1 with high affinity and blocks its ability to bind to PD-1 and cluster of differentiation CD80. The fragment crystallizable (Fc) domain of durvalumab contains a triple mutation in the constant domain of the IgG1 heavy chain that reduces binding to the complement component C1q and the Fc gamma receptors responsible for mediating antibody-dependent cell-mediated cytotoxicity (ADCC; Oganesyan, 2008).

In some embodiments, an inhibitor of PD-1 is a monoclonal antibody that binds to PD-1. This binding blocks the interaction with PD-L1 or inhibits and downstream signaling that results from the interaction between PD-1 and PD-L1. Non-limiting examples monoclonal antibodies that bind PD-1 include: nivolumab, pembrolizumab, pidilizumab, AMP-224, and TSR-042.

Any of the known anti-PD-1 agents provided in the following publications can be used: U.S. Pat. Nos. 7,101,550, 5,698,520, 6,808,710, 7,029,674, 7,794,710, 7,892,540, 8,008,449, 8,088,905, 8,163,503, 8,168,757, 8,354,509, 8,460,927, 8,609,089, 8,747,833, 8,779,105, 8,900,587, 8,952,136, 8,981,063, 8,993,731, 9,062,112, 9,067,999, 9,073,994, 9,084,776, 9,102,728, and 7,488,802; and U.S. Patent Publication Nos. US20020055139, US20140044738, each of which is herein incorporated by reference in its entirety. For example, monoclonal antibodies that bind PD-1 include: nivolumab, pembrolizumab, pidilizumab, AMP-224, and TSR-042.

In some embodiments, an inhibitor of PD(L)1 is a small molecule inhibitor. Non-limiting examples of small molecule inhibitors of PD(L)1 include: CA-170, BMS-1001, and BMS-1166. In some embodiments, a small molecule inhibitor of PD(L)1 is a natural product-derived PD-(L)1 inhibitor.

In some embodiments, a small molecule inhibitor of PD(L)1 decrease PD-L1 stability by regulating PD-L1 phosphorylation (e.g., metformin, EGF inhibitor(s), JAK inhibitors, or a combination thereof).

In some embodiments, a small molecule inhibitor of PD(L)1 decreases PD-L1 stability by regulating glycosylation (e.g., signal inhibitor, inhibitors of FKBP51S (e.g., SAFit 1 and SAFit 2), Curcumin, BMS-1166, or a combination thereof).

In some embodiments, a small molecule can be used to increase PD-L1 stability thereby further sensitizing tumor cells to the currently available immune checkpoint inhibitors. For example, two selective SMIs of CDK4/6, palbociclib and ribociclib, when used for the treatment of cancer cell lines, increased both PD-L1 stability and protein levels, thereby further sensitizing tumor cells to the currently available immune checkpoint inhibitors.

In some embodiments, the methods described herein can include at least two (e.g., at least three, at least four, at least five or at least six) PD-1/PD-L1 inhibitors (e.g., any of the PD-L1 inhibitors described herein).

In some embodiments, an inhibitor of PD-1/PD-L1 comprises one or more monoclonal antibodies that bind to PD-1 or PD-L1 and one or more small molecule inhibitors of PD-1/PD-L1.

In some embodiments, the methods described herein can include one or more PD(L)1 inhibitors (e.g., any of the PD-1/PD-L1 inhibitors described herein (e.g., small molecule or antibody) and one or more CTLA-4 inhibitors (e.g., any of the CTLA-4 inhibitors described herein (e.g., antibodies)).

### 7.4.2. CTLA-4 Inhibitor

In some embodiments, an immune checkpoint inhibitor (e.g., a first immune checkpoint inhibitor and a second immune checkpoint inhibitor) is an inhibitor of CTLA-4. As used herein, “CTLA-4” refers to cytotoxic T-lymphocyte associated protein 4. CTLA-4 is a member of the immunoglobulin superfamily and encodes a protein which transmits an inhibitory signal to T cells.

In some embodiments, an inhibitor of CTLA-4 includes an inhibitor that blocks the interaction between CTLA-4 and B7. In particular, CTLA-4 is an immune checkpoint molecule, which is up-regulated on activated T-cells. Without wishing to be bound by theory, an inhibitor anti-CTLA-4 can block the interaction of CTLA-4 with CD80/86 and switch off the mechanism of immune suppression and enable continuous stimulation of T-cells by DCs.

In some embodiments, an inhibitor of CTLA-4 is a monoclonal antibody that binds to CTLA-4. This binding blocks the interaction with B7 or inhibits and downstream signaling that results from the interaction between B7 and CTLA-4.

In some embodiments, the monoclonal antibody that binds CTLA-4 is selected from: tremelimumab, ipilimumab, quavonlimab (MK-1308), zalifrelimab (AGEN1884), GIGA-564, CBT-509, and AGEN1181.

Non-limiting examples of anti-CTLA-4 antibodies are ipilimumab (see U.S. Pat. Nos. 6,984,720, 7,452,535, 7,605,238, 8,017,114, and 8,142,778), tremelimumab (see U.S. Pat. Nos. 668,736, 7,109,003, 7,132,281, 7,411,057, 7,807,797, 7,824,679 and 8,143,379) and other anti-CTLA-4 antibodies, including single chain antibodies (e.g., see U.S. Pat. Nos. 5,811,097, 6,051,227 and 7,229,628, and US Patent Publication No. US20110044953), each of which are herein incorporated by reference in their entireties.

In some embodiments, the monoclonal antibody that binds to CTLA-4 is tremelimumab. Tremelimumab is a human IgG2 mAb being investigated as a cancer immunotherapeutic agent. Tremelimumab is expressed in NS0 (murine myeloma) cells and has an overall molecular weight of approximately 149 kDa. Tremelimumab is specific for human CTLA-4, with no cross-reactivity to related human proteins. Tremelimumab blocks the inhibitory effect of CTLA-4, and therefore enhances T cell activation. Tremelimumab shows minimal specific binding to Fc receptors, does not induce natural killer ADCC activity, and does not deliver inhibitory signals following plate-bound aggregation.

In some embodiments, an inhibitor of CTLA-4 is a small molecule inhibitor. Small molecule inhibitors of CTLA-4 include, without limitation, MDK 24720 and CCX6239. In some embodiments, the small molecule inhibitor of CTLA-4 is a natural product-derived CTLA-4 inhibitor.

In some embodiments, the methods described herein can include at least two (e.g., at least three, at least four, at least five or at least six) CTLA-4 inhibitors (e.g., any of the CTLA-4 inhibitors described herein).

In some embodiments, an inhibitor of CTLA-4 comprises one or more monoclonal antibodies that bind to CTLA-4 and one or more small molecule inhibitors of CTLA-4.

In some embodiments, the methods described herein two or more immune check point inhibitors. In some embodiments, the methods described herein include durvalumab and tremelimumab. A Phase 1b study was previously conducted to evaluate the safety, tolerability, pharmacokinetic (PK), immunogenicity, and antitumor activity of durvalumab in combination with tremelimumab in adult subjects with advanced NSCLC (see NCT02000947). In the NCT02000947 study, durvalumab was administered on a Q4W dosing schedule. In particular, durvalumab was administered Q4W for 13 doses and the Q2W dosing schedule had durvalumab administered Q2W for 26 doses. Doses tested range from 3 mg/kg to 20 mg/kg. In the NCT02000947 study for tremelimumab was administered Q4W for 6 doses and Q12W for 3 doses; Dose 7 was given at 4 weeks from Dose 6 and Dose 8 was given at 12 weeks from Dose 7. Doses tested range from 1 mg/kg to 10 mg/kg.

### 7.5. Dose and Dosing Schedule

### 7.5.1. Tebentafusp

In some embodiments, the method includes administering a therapeutically effective dose of tebentafusp to a melanoma patient. For example, when administered to the patient, therapeutically effective dose of tebentafusp is sufficient effect treatment (e.g., increase overall survival rate at 1 year).

In some embodiments, the method includes administering to a patient tebentafusp at a dose ranging from about 10 mcg to about 68 mcg (e.g., about 10 mcg to about 60 mcg, about 10 mcg to about 55 mcg, about 10 mcg to about 50 mcg, about 10 mcg to about 45 mcg, about 10 mcg to about 40 mcg, about 10 mcg to about 35 mcg, about 10 mcg to about 30 mcg, about 10 mcg to about 25 mcg, about 10 mcg to about 20 mcg, about 10 mcg to about 15 mcg, about 15 mcg to about 68 mcg, about 15 mcg to about 60 mcg, about 15 mcg to about 55 mcg, about 15 mcg to about 50 mcg, about 15 mcg to about 45 mcg, about 15 mcg to about 40 mcg, about 15 mcg to about 35 mcg, about 15 mcg to about 30 mcg, about 15 mcg to about 25 mcg, about 15 mcg to about 20 mcg, about 20 mcg to about 68 mcg, about 20 mcg to about 60 mcg, about 20 mcg to about 55 mcg, about 20 mcg to about 50 mcg, about 20 mcg to about 45 mcg, about 20 mcg to about 40 mcg, about 20 mcg to about 35 mcg, about 20 mcg to about 30 mcg, about 20 mcg to about 25 mcg, about 25 mcg to about 68 mcg, about 25 mcg to about 60 mcg, about 25 mcg to about 55 mcg, about 25 mcg to about 50 mcg, about 25 mcg to about 45 mcg, about 25 mcg to about 40 mcg, about 25 mcg to about 35 mcg, about 25 mcg to about 30 mcg, about 30 mcg to about 68 mcg, about 30 mcg to about 60 mcg, about 30 mcg to about 55 mcg, about 30 mcg to about 50 mcg, about 30 mcg to about 45 mcg, about 30 mcg to about 40 mcg, about 30 mcg to about 35 mcg, about 35 mcg to about 68 mcg, about 35 mcg to about 60 mcg, about 35 mcg to about 55 mcg, about 35 mcg to about 50 mcg, about 35 mcg to about 45 mcg, about 35 mcg to about 40 mcg, about 40 mcg to about 68 mcg, about 40 mcg to about 60 mcg, about 40 mcg to about 55 mcg, about 40 mcg to about 50 mcg, about 40 mcg to about 45 mcg, about 45 mcg to about 68 mcg, about 45 mcg to about 60 mcg, about 45 mcg to about 55 mcg, about 45 mcg to about 50 mcg, about 50 mcg to about 68 mcg, about 50 mcg to about 60 mcg, about 50 mcg to about 55 mcg, about 55 mcg to about 68 mcg, about 55 mcg to about 60 mcg, or about 60 mcg to about 68 mcg).

In some embodiments, the method includes administering to a patient tebentafusp at a dose ranging from about 10 mcg to about 50 mcg (e.g., any of the values or subranges therein).

In some embodiments, the method includes administering to a patient tebentafusp at a dose ranging from about 68 mcg to about 200 mcg (e.g., about 68 mcg to about 180 mcg, about 68 mcg to about 160 mcg, about 68 mcg to about 140 mcg, about 68 mcg to about 120 mcg, about 68 mcg to about 100 mcg, about 68 mcg to about 80 mcg, about 80 mcg to about 200 mcg, about 80 mcg to about 180 mcg, about 80 mcg to about 160 mcg, about 80 mcg to about 140 mcg, about 80 mcg to about 120 mcg, about 80 mcg to about 100 mcg, about 100 mcg to about 200 mcg, about 100 mcg to about 180 mcg, about 100 mcg to about 160 mcg, about 100 mcg to about 140 mcg, about 100 mcg to about 120, about 120 mcg to about 200 mcg, about 120 mcg to about 180 mcg, about 120 mcg to about 160 mcg, about 120 mcg to about 140 mcg, about 140 mcg to about 200 mcg, about 140 mcg to about 180 mcg, about 140 mcg to about 160 mcg, about 160 mcg to about 200 mcg, about 160 mcg to about 180 mcg, or about 180 mcg to about 200 mcg).

In some embodiments, the method includes administering to a patient tebentafusp at a dose of about any one of 0.1 mcg, 0.5 mcg, 1.0 mcg, 1.5 mcg, 2.0 mcg, 2.5 mcg, 3.0 mcg, 3.5 mcg, 4.0 mcg, 4.5 mcg, 5.0 mcg, 5.5 mcg, 6.0 mcg, 6.5 mcg, 7.0 mcg, 7.5 mcg, 8.0 mcg, 9.0 mcg, 10 mcg, 11 mcg, 12 mcg, 13 mcg, 14 mcg, 15 mcg, 16 mcg, 17 mcg, 18 mcg, 19 mcg, 20 mcg, 21 mcg, 22 mcg, 23 mcg, 24 mcg, 25 mcg, 26 mcg, 27 mcg, 28 mcg, 29 mcg, 30 mcg, 31 mcg, 32 mcg, 33 mcg, 34 mcg, 35 mcg, 36 mcg, 37 mcg, 38 mcg, 39 mcg, 40 mcg, 41 mcg, 42 mcg, 43 mcg, 44 mcg, 45 mcg, 46 mcg, 47 mcg, 48 mcg, 49 mcg, 50 mcg, 51 mcg, 52 mcg, 53 mcg, 54 mcg, 55 mcg, 56 mcg, 57 mcg, 58 mcg, 59 mcg, 60 mcg, 61 mcg, 62 mcg, 63 mcg, 64 mcg, 65 mcg, 66 mcg, 67 mcg, 68 mcg, 69 mcg, 70 mcg, 71 mcg, 72 mcg, 73 mcg, 74 mcg, 75 mcg, 76 mcg, 77 mcg, 78 mcg, 79 mcg, 80 mcg, 81 mcg, 82 mcg, 83 mcg, 84 mcg, 85 mcg, 86 mcg, 87 mcg, 88 mcg, 89 mcg, 90 mcg, 91 mcg, 92 mcg, 93 mcg, 94 mcg, 95 mcg, 96 mcg, 97 mcg, 98 mcg, 99 mcg, 100 mcg, 105 mcg, 110 mcg, 115 mcg, 120 mcg, 125 mcg, 130 mcg, 135 mcg, 140 mcg, 145 mcg, 150 mcg, 155 mcg, 160 mcg, 165 mcg, 170 mcg, 175 mcg, 180 mcg, 185 mcg, 190 mcg, 195 mcg, 200 mcg or more.

In some embodiments, the one or more immune checkpoint inhibitor is administered after a dose escalation regimen for tebentafusp. In some embodiments, the dose escalation regimen for tebentafusp is an intra-patient dose escalation regimen as described in U.S. Pat. Publication No. 2020/0040055A1, which is herein incorporated by reference in its entirety. In such embodiments, the intra-patient dose escalation regimen provides improved tolerability for treating gp100 positive tumors with a bi-specific therapeutic employing a T cell redirection mediated mechanism-of-action (i.e., tebentafusp). A non-limiting example of an intra-patient dose escalation for tebentafusp includes administering: at least one first dose of tebentafusp in the range of from 10-30 μg; (b) at least one second dose of tebentafusp in the range of from 20-40 μg, wherein the second dose is higher than the first dose; and then (c) at least one dose of at least 50 μg of tebentafusp.

In some embodiments, tebentafusp is administered to a melanoma patient about once weekly. In some embodiments, tebentafusp is administered to a melanoma patient every 5 to 10 days, every 5 to 9 days, every 5 to 8 days, every 5 to 7 days, every 5 to 6 days, every 6 to 10 days, every 6 to 9 days, every 6 to 8 days, every 6 to 7 days, every 7 to 10 days, every 7 to 9 days, every 7 to 8 days, every 8 to 10 days, every 8 to 9 days, or every 9 to 10 days. In some embodiments, tebentafusp is administered to a melanoma patient every 7 days.

In some embodiments, tebentafusp is administered to a melanoma patient about once every two weeks, about once every three weeks, about once every four weeks, about once every five weeks, about once every six weeks, about once every seven weeks, or about once every eight weeks.

### 7.5.2. Immune Checkpoint Inhibitor

In some embodiments, the method includes administering a therapeutically effective dose of tebentafusp to a melanoma patient. For example, when administered to the patient in a combination therapy with an immune checkpoint inhibitor, a therapeutically effective dose of tebentafusp is sufficient effect treatment (e.g., increase overall survival rate at 1 year).

As noted above, in one embodiment, a checkpoint inhibitor is first administered to a patient after a dose escalation regimen (e.g., an intra-patient dose escalation regimen) for tebentafusp. For example, the checkpoint inhibitor is first administered to a patient after a dose escalation regimen for tebentafusp that occurs over a 1, 2, 3, 4, 5, 6, 7, 8, 9, 10, 11, 12, 13, 14, 15, 16, 17, 18, 19, 20, 21, 22, 23, 24, 25, 26, 27 or 28 day period. In one embodiment, the checkpoint inhibitor is first administered to a patient after a dose escalation regimen that occurs over a 15 day period. For example, the checkpoint inhibitor is administered 15 days after the first administration of tebentafusp but following a dose escalation regimen that includes additional doses of tebentafusp during the 15 day period. In another embodiment, the checkpoint inhibitor is first administered to a patient after a dose escalation regimen that occurs over a 22 day period. For example, the checkpoint inhibitor is administered 22 days after the first administration of tebentafusp but following a dose escalation regimen that includes additional doses of tebentafusp during the 22 day period.

In some embodiments, the first immune checkpoint inhibitor, the second immune checkpoint inhibitor, or both, are first administered about 15 days after the first administration of the tebentafusp.

In some embodiments, the method includes concurrently administering to a melanoma patient a first immune checkpoint inhibitor and second immune checkpoint inhibitor. In some embodiments, the method includes administering to a melanoma patient a first immune checkpoint inhibitor and second immune checkpoint inhibitor on the same day.

In some embodiments, the method includes separately administering to a melanoma patient a first immune checkpoint inhibitor and a second immune checkpoint inhibitor. In some embodiments, the method includes administering to a melanoma patient a first immune checkpoint inhibitor and a second immune checkpoint inhibitor at least 1 day, 2 days, 3 days, 4 days, 5 days, 6 days, or 7 days apart. In some embodiments, the method includes administering to a melanoma patient a first immune checkpoint inhibitor and a second immune checkpoint inhibitor at least 2 weeks, 3 weeks, 4 weeks, 5 weeks, 6 weeks, 7 weeks, or 8 weeks apart.

### 7.5.1. PD(L)1 Inhibitor

In some embodiments, the method includes administering a therapeutically effective dose of a PD(L)1 inhibitor to a melanoma patient. For example, when administered to the patient in a combination therapy with tebentafusp, a therapeutically effective dose of a PD(L)1 inhibitor is sufficient effect treatment (e.g., increase overall survival rate at 1 year).

In some embodiments, the PD(L)1 inhibitor is durvalumab, where durvalumab is administered to the melanoma patient at a dose ranging from about 1 mg/kg to about 20 mg/kg (e.g., about 1 mg/kg to about 18 mg/kg, about 1 mg/kg to about 16 mg/kg, about 1 mg/kg to about 14 mg/kg, about 1 mg/kg to about 12 mg/kg, about 1 mg/kg to about 10 mg/kg, about 1 mg/kg to about 8 mg/kg, about 1 mg/kg to about 6 mg/kg, about 1 mg/kg to about 4 mg/kg, about 1 mg/kg to about 2 mg/kg, about 2 mg/kg to about 20 mg/kg, about 2 mg/kg to about 18 mg/kg, about 2 mg/kg to about 16 mg/kg, about 2 mg/kg to about 14 mg/kg, about 2 mg/kg to about 12 mg/kg, about 2 mg/kg to about 10 mg/kg, about 2 mg/kg to about 8 mg/kg, about 2 mg/kg to about 6 mg/kg, about 2 mg/kg to about 4 mg/kg, about 4 mg/kg to about 20 mg/kg, about 4 mg/kg to about 18 mg/kg, about 4 mg/kg to about 16 mg/kg, about 4 mg/kg to about 14 mg/kg, about 4 mg/kg to about 12 mg/kg, about 4 mg/kg to about 10 mg/kg, about 4 mg/kg to about 8 mg/kg, about 4 mg/kg to about 6 mg/kg, about 6 mg/kg to about 20 mg/kg, about 6 mg/kg to about 18 mg/kg, about 6 mg/kg to about 16 mg/kg, about 6 mg/kg to about 14 mg/kg, about 6 mg/kg to about 12 mg/kg, about 6 mg/kg to about 10 mg/kg, about 6 mg/kg to about 8 mg/kg, about 8 mg/kg to about 20 mg/kg, about 8 mg/kg to about 18 mg/kg, about 8 mg/kg to about 16 mg/kg, about 8 mg/kg to about 14 mg/kg, about 8 mg/kg to about 12 mg/kg, about 8 mg/kg to about 10 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 20 mg/kg, about 14 mg/kg to about 18 mg/kg, about 14 mg/kg to about 16 mg/kg, about 16 mg/kg to about 20 mg/kg, about 16 mg/kg to about 18 mg/kg, or about 18 mg/kg to about 20 mg/kg).

In some embodiments, the PD(L)1 inhibitor is durvalumab, where durvalumab is administered to the melanoma patient at a dose of about ≥10 mg/kg (e.g., about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg or more).

In the PD(L)1 inhibitor is durvalumab, where durvalumab is administered to the melanoma patient at a dose of about <10 mg/kg (e.g., about 9 mg/kg, about 8 mg/kg, about 7 mg/kg, about 6 mg/kg, about 5 mg/kg, about 4 mg/kg, about 3 mg/kg, about 2 mg/kg or about 1 mg/kg).

In some embodiments, the PD(L)1 inhibitor is pembrolizumab, where pembrolizumab is administered to the melanoma patient at a dose ranging from about 200 mg IV about every 3 weeks. In some embodiments, the PD(L)1 inhibitor is pembrolizumab, where pembrolizumab is administered to the melanoma patient at a dose ranging from about 400 mg IV about every 6 weeks.

In some embodiments, the PD(L)1 inhibitor is nivolumab, where nivolumab is indicated for melanoma in patients with lymph node involvement or metastatic disease who have undergone complete resection, in the adjuvant setting. In some embodiments, nivolumab is used in combination with ipilimumab. In some embodiments, the PD(L)1 inhibitor is nivolumab, where nivolumab is administered to the melanoma patient at a dose ranging from about 240 mg IV about every 2 weeks. In some embodiments, the PD(L)1 inhibitor is nivolumab, where nivolumab is administered to the melanoma patient at a dose ranging from about 480 mg IV about every 4 weeks. In some embodiments, the PD(L)1 inhibitor is nivolumab, where nivolumab is administered to the melanoma patient at a dose ranging from about 1 mg/kg (e.g., when used in combination with ipilimumab).

In some embodiments, the PD(L)1 inhibitor is dostarlimab, where dostarlimab is administered to the melanoma patient at a dose ranging from about 500 mg IV about every 3 weeks (doses 1-4) and then 1000 mg IV every 6 weeks (dose 5). In some embodiments, the PD(L)1 inhibitor is dostarlimab, where dostarlimab is administered to the melanoma patient at a dose ranging from about ≤10 mg/day (or equivalent).

In some embodiments, the PD(L)1 inhibitor is cemiplimap, where cemiplimap is administered to the melanoma patient at a dose ranging from about 350 mg IV about every 3 weeks.

In some embodiments, the PD(L)1 inhibitor is atezolizumab, where atezolizumab is indicated for unresectable or metastatic BRAF V600 mutation-positive melanoma in combination. In some cases, atezolizumab with cobimetinib and vemurafenib. In some embodiments, the PD(L)1 inhibitor is atezolizumab, where atezolizumab is administered to the melanoma patient at a dose ranging from about 840 mg IV about every 3 weeks.

avelumab (Bavencio) In some embodiments, the PD(L)1 inhibitor is avelumab, where avelumab is administered to the melanoma patient at a dose ranging from about 800 mg IV about every 2 weeks.

### 7.5.2. CTLA-4 Inhibitor

In some embodiments, the method includes administering a therapeutically effective dose of a CTLA-4 inhibitor to a melanoma patient. For example, when administered to the patient in a combination therapy with tebentafusp, a therapeutically effective dose of a CTLA-4 inhibitor is sufficient effect treatment (e.g., increase overall survival rate at 1 year).

In some embodiments, a CTLA-4 inhibitor is tremelimumab, where tremelimumab is administered to the melanoma patient at a dose ranging from about 0.5 mg/kg to about 10 mg/kg (e.g., about 0.5 mg/kg to about 9 mg/kg, about 0.5 mg/kg to about 8 mg/kg, about 0.5 mg/kg to about 7 mg/kg, about 0.5 mg/kg to about 6 mg/kg, about 0.5 mg/kg to about 5 mg/kg, about 0.5 mg/kg to about 4 mg/kg, about 0.5 mg/kg to about 3 mg/kg, about 0.5 mg/kg to about 2.0 mg/kg, about 0.5 mg/kg to about 1.0 mg/kg, about 1.0 mg/kg to about 10 mg/kg, about 1.0 mg/kg to about 9 mg/kg, about 1.0 mg/kg to about 8 mg/kg, about 1.0 mg/kg to about 7 mg/kg, about 1.0 mg/kg to about 6 mg/kg, about 1.0 mg/kg to about 5 mg/kg, about 1.0 mg/kg to about 4 mg/kg, about 1.0 mg/kg to about 3 mg/kg, about 1.0 mg/kg to about 2.0 mg/kg, about 2.0 mg/kg to about 10 mg/kg, about 2.0 mg/kg to about 9 mg/kg, about 2.0 mg/kg to about 8 mg/kg, about 2.0 mg/kg to about 7 mg/kg, about 2.0 mg/kg to about 6 mg/kg, about 2.0 mg/kg to about 5 mg/kg, about 2.0 mg/kg to about 4 mg/kg, about 2.0 mg/kg to about 3 mg/kg, about 3.0 mg/kg to about 10 mg/kg, about 3.0 mg/kg to about 9 mg/kg, about 3.0 mg/kg to about 8 mg/kg, about 3.0 mg/kg to about 7 mg/kg, about 3.0 mg/kg to about 6 mg/kg, about 3.0 mg/kg to about 5 mg/kg, about 3.0 mg/kg to about 4 mg/kg, about 4.0 mg/kg to about 10 mg/kg, about 4.0 mg/kg to about 9 mg/kg, about 4.0 mg/kg to about 8 mg/kg, about 4.0 mg/kg to about 7 mg/kg, about 4.0 mg/kg to about 6 mg/kg, about 4.0 mg/kg to about 5 mg/kg, about 5.0 mg/kg to about 10 mg/kg, about 5.0 mg/kg to about 9 mg/kg, about 5.0 mg/kg to about 8 mg/kg, about 5.0 mg/kg to about 7 mg/kg, about 5.0 mg/kg to about 6 mg/kg, about 6.0 mg/kg to about 10 mg/kg, about 6.0 mg/kg to about 9 mg/kg, about 6.0 mg/kg to about 8 mg/kg, about 6.0 mg/kg to about 7 mg/kg, about 7.0 mg/kg to about 10 mg/kg, about 7.0 mg/kg to about 9 mg/kg, about 7.0 mg/kg to about 8 mg/kg, about 8.0 mg/kg to about 10 mg/kg, about 8.0 mg/kg to about 9 mg/kg, or about 9 mg/kg to about 10 mg/kg).

### 7.5.3. Combination Dosing Schedule

In some embodiments, where the method includes administering tebentafusp and durvalumab, or tebentafusp and tremelimumab, the immune checkpoint inhibitor therapy is administered before administration of tebentafusp. For example, in the doublet arms (Arms 1 and 2) durvalumab or tremelimumab were administered first (1 hour infusion), followed by a break of 60 minutes (e.g., up to 3 hours duration if clinically indicated) before administration of tebentafusp (e.g., 15 to 20 minute infusion).

In some embodiments, where the method includes administering tebentafusp and durvalumab or tebentafusp and tremelimumab, tebentafusp is administered before the immune checkpoint inhibitor therapy is administered.

In some embodiments, where the method includes administering tebentafusp, durvalumab, and tremelimumab, both of the immune checkpoint inhibitors are administered before administration of tebentafusp. In some embodiments, durvalumab is administered before administration of tremelimumab. In some embodiments, tremelimumab is administered before administration of durvalumab. In one non-limiting example, for Arm 3, tremelimumab is administered first; the durvalumab infusion is started approximately 1 hour (maximum 2 hours) after the end of the tremelimumab infusion, followed by a break of 60 minutes (e.g., up to 3 hours duration if clinically indicated) before administration of tebentafusp (e.g., 15 to 20 minute infusion).

In some embodiments, where the method includes administering tebentafusp, durvalumab, and tremelimumab, tebentafusp is administered before administration of the immune checkpoint inhibitors. In some embodiments, durvalumab is administered before administration of tremelimumab. In some embodiments, tremelimumab is administered before administration of durvalumab.

### 7.6. Routes of Administration

In some embodiments, the route of administration of tebentafusp, the first immune checkpoint inhibitor, the second immune checkpoint inhibitor, or any combination thereof, depend on the nature (e.g., stage) of the melanoma.

In some embodiments, the route of administration includes, but is not limited to, intratumoral, intravesical, intramuscular, intraperitoneal, intravenous, intra-arterial, intrapleural, subcutaneous, and epidermal routes, or be delivered into lymph glands, body spaces, organs or tissues known to contain such live cancer cells (such as intrahepatic or intrapancreatic injections). In some embodiments, the administration is carried out by direct injection of the agent(s) into the tumor. In some embodiments, the administration is carried out by direct injection of the agent(s) to a site close to the tumor cells.

In some embodiments, the route of administration of tebentafusp, the first immune checkpoint inhibitor, the second immune checkpoint inhibitor, or any combination thereof, includes intravenous infusion, subcutaneous infusion, intramuscular infusion, enteral administration, inhalation, or intranasal.

In some embodiment, the tebentafusp is administered via intravenous infusion, subcutaneous infusion, intramuscular infusion, enteral administration, inhalation, or intranasal.

In some embodiments, the first immune checkpoint inhibitor, the second immune checkpoint inhibitor, or both are administered via intravenous infusion, subcutaneous infusion, intramuscular infusion, enteral administration, inhalation, or intranasal.

In some embodiments, the administration of the tebentafusp, the first immune checkpoint inhibitor, the second immune checkpoint inhibitor, or any combination thereof, is carried out by injection into the skin tissue having the melanoma tumor. In some embodiments, the administration of the administration of the tebentafusp, the first immune checkpoint inhibitor, the second immune checkpoint inhibitor, or any combination thereof, is carried out by injection directly into the melanoma tumor. In some embodiments, the administration of the administration of the tebentafusp, the first immune checkpoint inhibitor, the second immune checkpoint inhibitor, or any combination thereof, is carried out by injection directly into metastatic sites of the melanoma tumor.

### 7.7. Patient Population

In some embodiments, the patient has or is suspected of having melanoma. In some embodiments, the melanoma is cutaneous melanoma, uveal melanoma, superficial spreading melanoma, lentigo maligna melanoma, nodular melanoma, mucosal melanoma, polypoid melanoma, desmoplastic melanoma, amelanotic melanoma, soft-tissue melanoma, or acral ientiginous melanoma. In some embodiments, the melanoma is at any of stage I, II, III, or IV, according to the American Joint Committee on Cancer (AJCC) staging groups. In some embodiments, the melanoma is recurrent. In some embodiments, the melanoma is refractory. In some embodiments, the patient relapse with melanoma.

In some embodiments, the patient has or is suspected of having cutaneous melanoma. In some embodiments, the patient has or is suspected of having metastatic cutaneous melanoma.

In some embodiments, the patient has melanoma that is refractory or resistant. In some embodiments, the patient has a melanoma that is refractory or resistant following radiation or treatment with a chemotherapeutic agent (e.g., dacarbazine and temozolamide), an immunotherapeutic agent (e.g., interleukin-2 (IL-2) and interferon (IFN)), an immune checkpoint inhibitors (e.g., anti-PD(L)1 agents (e.g., durvalumab, atezolizumab, BMS-936559, avelumab, pembrolizumab, nivolumab, dostarlimab, and cemiplimap), and anti-CTLA-4 (e.g., ipilimumab and tremelimumab), BRAF inhibitors (e.g., vemurafenib and dabrafenib), MEK inhibitors (e.g., trametinib), TGF-β inhibitors (e.g., galunisertib), MET kinase inhibitors (e.g., merestinib), or a combination thereof.

In some embodiments, the patient has a melanoma that is refractory or resistant following a prior checkpoint inhibitor treatment. In some embodiments, the patient was previously treated with a PD(L)1 inhibitor. In one embodiment, the patient received prior anti-PD1 but it was not most recent therapy prior to enrollment (i.e., “remote,” see FIG. 12). In another embodiment, the PD(L)1 inhibitor was the most recent cancer therapy administered to the patient prior to enrollment (i.e., “immediately prior anti-PD(L)1,” see FIG. 12).

In some embodiments, the melanoma patient has a BRAF mutation. In such embodiments, the patient was previously treated with an approved BRAF-based therapy.

In some embodiments, the melanoma patient is positive for a human leukocyte antigen serotype of HLA-A*02. In some embodiments, the melanoma patient has a YLEPGPVTA (SEQ ID NO: 3) peptide -HLA-A*02 complex. In such cases, tebentafusp binds the YLEPGPVTA (SEQ ID NO: 3) peptide -HLA-A*02 complex and induces killing via the recruitment of both tumor specific and/or non-tumor specific T cells.

### 8. EXAMPLES

### 8.1. Example 1: Phase 1b/2 Study of the Combination of Tebentafusp with Durvalumab and/or Tremelimumab in Cutaneous Melanoma

### 8.1.1. Study Description

The study described herein is a Phase Ib/II, multi-center, open-label study of tebentafusp (IMCgp100) as a single agent and in combination with durvalumab and/or tremelimumab in metastatic cutaneous melanoma. The purpose of this on-going study is to characterize the safety, tolerability, pharmacokinetics (PK), pharmacodynamics, and to evaluate the anti-tumor activity of tebentafusp (IMCgp100) in combination with durvalumab (programmed death-ligand 1 [PD-L1] inhibitor), tremelimumab (CLTA-4 inhibitor), and the combination of durvalumab with tremelimumab compared to single-agent tebentafusp (IMCgp100) alone. The study enrolled patients who have metastatic melanoma that is refractory to treatment with an anti-PD-1 inhibitor in the metastatic setting. The study also evaluated the safety, tolerability, and anti-tumor activity of tebentafusp (IMCgp100) monotherapy in patients with advanced non-uveal melanoma who progressed on prior PD-1 inhibitors approved for the treatment of advanced melanoma; patients with BRAF mutations must be refractory to approved BRAF-based therapy.

As noted above, recent biologic evidence indicates that optimal responses to programmed cell death-1 (PD-1) directed therapy require the presence of CD8⁺ T cells in the tumor microenvironment and thus therapies such as tebentafusp (IMCgp100) that recruit these effector cells to the tumor may overcome pre-existing resistance to checkpoint blockade. This emerging biology of checkpoint inhibitor resistance suggests the combination of tebentafusp (IMCgp100) with checkpoint inhibition may have enhanced activity in patients with pre-existing resistance.

### 8.1.2. Study Design

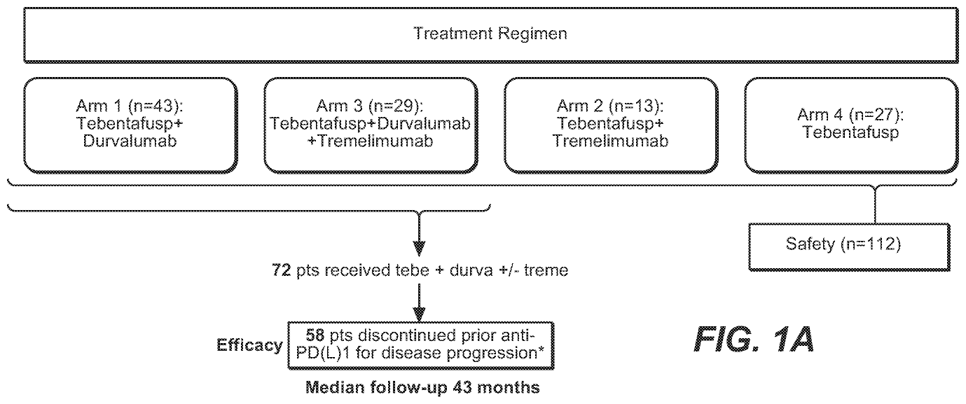

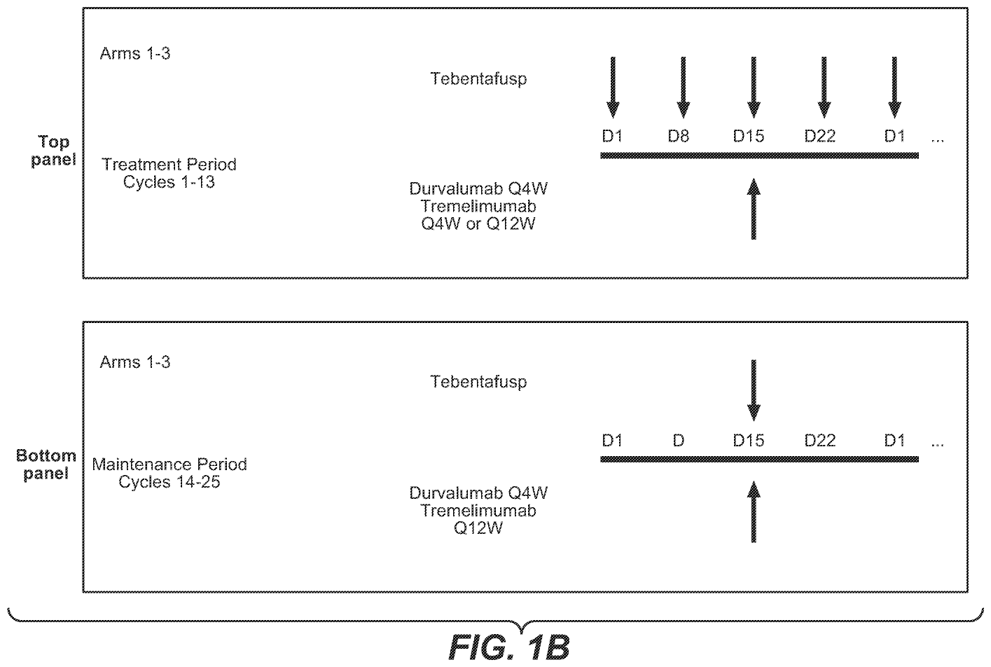

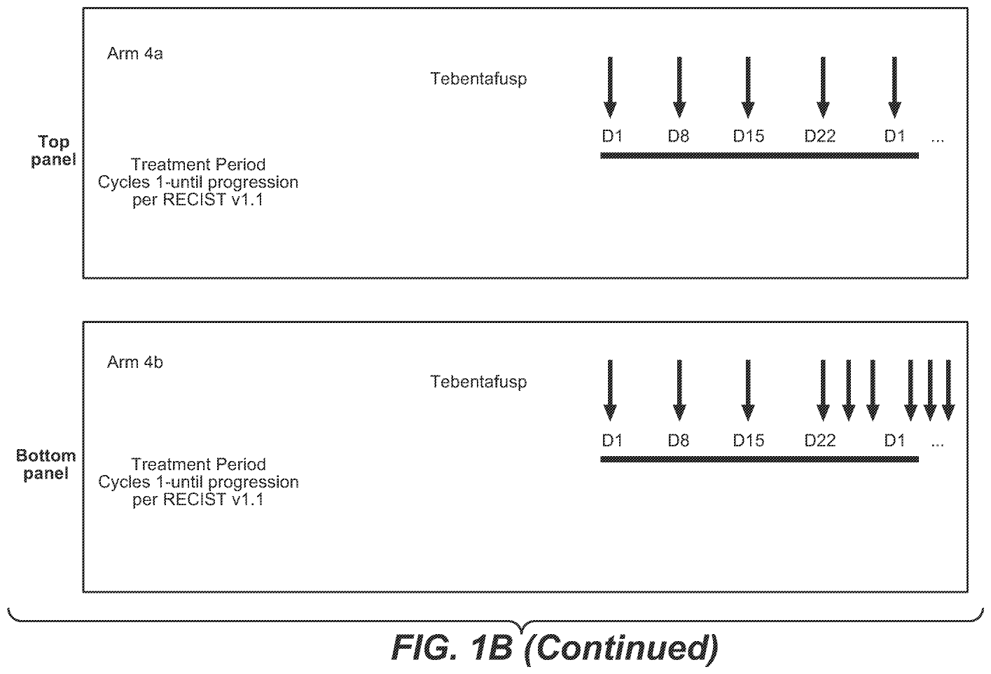

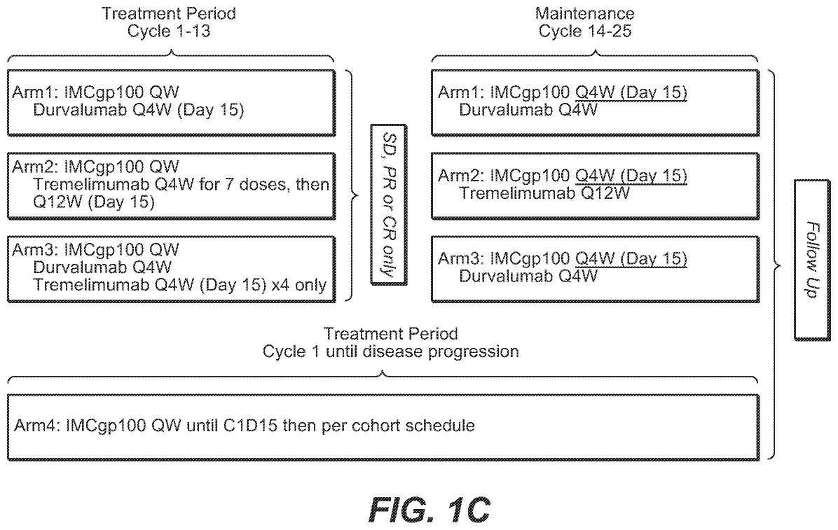

Enrollment included 312 participants. Experimental Arms 1-4 are as described below and in FIGS. 1A-1C. As described below, QW=once weekly; Q #W=once every #weeks; C#D #=Cycle #Day #; and RP2D=recommended Phase II dose.

Experimental Arm One: Participants received tebentafusp and durvalumab. Tebentafusp (i.e., soluble gp100-specific T cell receptor with anti-CD3 scFV) was administered intravenously (IV) at a dose ranging from about 10 mcg to about 68 mcg. Tebentafusp was administered IV about once weekly. Durvalumab (i.e., an anti-PD(L)1 monoclonal antibody) was administered IV at a dose ranging from about 1 mg/kg to about 20 mg/kg. The durvalumab was first administered IV about 15 days after the first administration of the tebentafusp. Thereafter, the durvalumab was administered IV about once every four weeks.

Experimental Arm Two: Participants received tebentafusp and tremelimumab. Tebentafusp (i.e., soluble gp100-specific T cell receptor with anti-CD3 scFV) was administered IV at a dose ranging from about 10 mcg to about 50 mcg. Tebentafusp was administered IV about once weekly. Tremelimumab (i.e., an anti-CTLA-4 monoclonal antibody) was administered IV at a dose ranging from about 0.5 mg/kg to about 10 mg/kg. The tremelimumab was first administered IV about 15 days after the first administration of the Tebentafusp. Thereafter, the tremelimumab was administered IV about once every four weeks.

Experimental Arm Three: Participants received tebentafusp and durvalumab and tremelimumab. Tebentafusp (i.e., soluble gp100-specific T cell receptor with anti-CD3 scFV) was administered IV at a dose ranging from about 10 mcg to about 50 mcg. Tebentafusp was administered IV about once weekly. Durvalumab (i.e., an anti-PD(L)1 monoclonal antibody) was administered IV at a dose ranging from about 1 mg/kg to about 20 mg/kg. The durvalumab was first administered IV about 15 days after the first administration of the tebentafusp. Thereafter, the durvalumab was administered IV about once every four weeks. Tremelimumab (i.e., an anti-CTLA-4 monoclonal antibody) was administered IV at a dose ranging from about 0.5 mg/kg to about 10 mg/kg. The was first administered IV about 15 days after the first administration of the Tebentafusp. Thereafter, the tremelimumab was administered IV about once every four weeks.

Experimental Arm Four: Participants received tebentafusp (single agent). Tebentafusp (i.e., soluble gp100-specific T cell receptor with anti-CD3 scFV) was administered IV at a dose ranging from about 68 mcg to about 200 mcg. Tebentafusp was administered IV about once weekly. Arm 4 consisted of two sub-arms in patients with advanced, non-uveal melanoma: arm 4a (tebentafusp dose escalation above 68 mcg (MTD defined by study IMCgp100-102) and arm 4b (tebentafusp exposure escalation investigating 3 times a week dosing of tebentafusp).

Each of the 3 combination treatment arms (arms 1, 2, and 3) followed the same dosing regimen over the first cycle (see, e.g., top panel of FIG. 1B “Treatment Period Cycles 1-13”). In this study, a cycle consisted of 4 weeks. In Cycle 1, patients received 2 weekly doses of tebentafusp, commencing with C1D1 and C1D8. Beginning on C1D15, patients initiated tebentafusp and checkpoint combination therapy with the assigned inhibitor (Arm 1 durvalumab, Arm 2 tremelimumab, or in Arm 3, the combination of durvalumab and tremelimumab) followed by the administration of the weekly dose of tebentafusp. In contrast to the 3 combination arms, patients assigned to Arm 4 did not receive checkpoint inhibitor combination therapy and received only tebentafusp.

The treatment schedule in each combination arm was divided into 2 periods: (1) the Treatment period with weekly tebentafusp and checkpoint inhibition lasting Cycles 1-13, and (2) the Maintenance period with reduced scheduling over Cycles 14-25, open for patients with evidence of disease control after the first 13 cycles in the Treatment period (i.e., or [partial response (PR) or complete response (CR)] or stable disease, SD) (see FIG. 1B and FIG. 1C).

### 8.1.3. Dosing Regimens

The dosing regimens of tebentafusp as a single agent and in combination with durvalumab and/or tremelimumab were as follows.

All patients received the tebentafusp weekly regimen in Arms 1-3 for a total of 13 cycles in the Treatment period. Two single-agent doses of Tebentafusp were administered at C1D1 and C1D8 followed by combination dosing with the checkpoint inhibitor at C1D15. In the Maintenance period, tebentafusp dosing was reduced to Q4W (Day 15 of each cycle).

Durvalumab in Arms 1 and 3 was given on Q4W (on Day 15 of each cycle) until treatment discontinuation. In doublet Arm 2, tremelimumab was dosed Q4W (on Day 15 of each cycle) for 7 doses (Cycles 1-7) followed by dosing every Q12W until treatment discontinuation. In Arm 3 (triplet), durvalumab was dosed Q4W (on Day 15 of each cycle, similar to Arm 1) until treatment discontinuation and tremelimumab was administered every Q4W for 4 doses only on Day 15 of Cycles 1-4. Tremelimumab dosing in Arm 3 (but not Arm 2) was stopped after the fourth dose is administered.

In Arms 1, 2 and 3, any patient with disease control at the completion of the first 13 cycles (the Treatment period; disease control is defined as SD, or an OR of PR or CR) entered the Maintenance period (Cycles 14-25). During Maintenance, dosing was continued with a reduced schedule of tebentafusp with 1 dose per cycle, Q4W (Day 15 of each cycle, coincident with the dosing of Q4W durvalumab in Arms 1 and 3 and Q12W tremelimumab in Arm 2). Patients who completed Cycle 25 therapy was stopped, and these patients entered the Follow-up period. Patients with disease that progressed within approximately 6 months of completing Cycle 25 were offered the option to re-start treatment for an additional 25 cycles. In Arm 4, all patients received tebentafusp weekly on C1D1, C1D8, and C1D15. On C1D22, patients received tebentafusp at the dose and schedule defined by the cohort.

### 8.1.3.1 Intra-patient Escalation in Phase Ib

In Arms 1-3, patients enrolled in lower dosing cohorts in each individual arm of treatment were provided the option to increase the dose of tebentafusp to the next dose level that has been declared as tolerated (tremelimumab and durvalumab doses were escalated for patients enrolled in individual cohorts). Patients enrolled in a lower dose cohorts could increase the dose by 1 dose level, provided they have been treated with the combination for a minimum of 12 weeks at the assigned dose level without severe toxicity, and the dose 1 level higher had been deemed as tolerated at the DETC. Patients receiving the next higher dose level of tebentafusp received the escalated dose of tebentafusp beginning with Day 1 of the next cycle of treatment, and patients must be hospitalized for the first escalated dose of tebentafusp.

### 8.1.4. Treatment Periods

The treatment regimen for patients included up to 4 periods: the initial Treatment period (Arms 1-3: lasting Cycles 1-13; Arm 4: lasting until progression) and, for patients in Arm 1-3 who achieved disease control (SD or OR [PR or CR]) at the end of Cycle 13, these patients had the option of entering the Maintenance period (Cycles 14-25). Patients enrolled in Arm 4 were continue in the Treatment period until treatment has discontinued for any reason. All patients entered the Follow-up period after treatment has discontinued for any reason.

The Pre-screening period began once a patient has signed the Pre-screening Informed Consent Form (ICF) and concludes with the HLA-A2 testing results. A blood test was used to determine whether the patient was HLA-A*0201 positive (eligible), or a non-HLA-A*0201 subtype (not eligible; e.g., *0202, *0203, *0204, *0205, etc), or HLA-A2 negative (not eligible). Patients were designated as pre-screening failure (negative test result or, if positive, excluded HLA subtype, or the patient chooses not to enter the study), or eligible for the study (HLA-A*0201 positive). Patients who were re-screened for the trial did need not to return to the pre-screen period once HLA results are known. Patients known to be HLA-A*0201 positive by prior testing done outside the study completed the pre-screening and screening periods in parallel.

The Screening period began once a patient signed the Main Study ICF and concluded with either a screening failure or study dosing on C1D1. During the Screening period, patients were evaluated against the study inclusion and exclusion criteria and all screening procedures and observations are performed. The screening window for all procedures was 21 days, other than imaging studies which had a 28-day window.

Treatment period: Cycle 1 for all patients in the 3 combination arms (Arms 1-3) consisted of single-agent dosing of tebentafusp for 2 weeks on C1D1 and C1D8. On C1D15, dosing of the combination checkpoint inhibitor (Arms 1-3, durvalumab and/or tremelimumab) was commenced. The checkpoint inhibitor regimen was administered first in the clinic, and tebentafusp was administered after completion of the checkpoint inhibitor (for details of combination administration). In Arms 1, 2 and 3, tebentafusp was administered weekly for the 13 cycles of the Treatment period. Patients enrolled in Arms 1, 2 and 3 in disease control at the end of 13 cycles, had the option of entering a reduced schedule of treatment in the Maintenance period. Patients enrolled in Arm 4 received escalating weekly doses of tebentafusp for the first three weeks (C1D1, C1D8 and C1D15) after which they received their allocated cohort dose level and schedule.

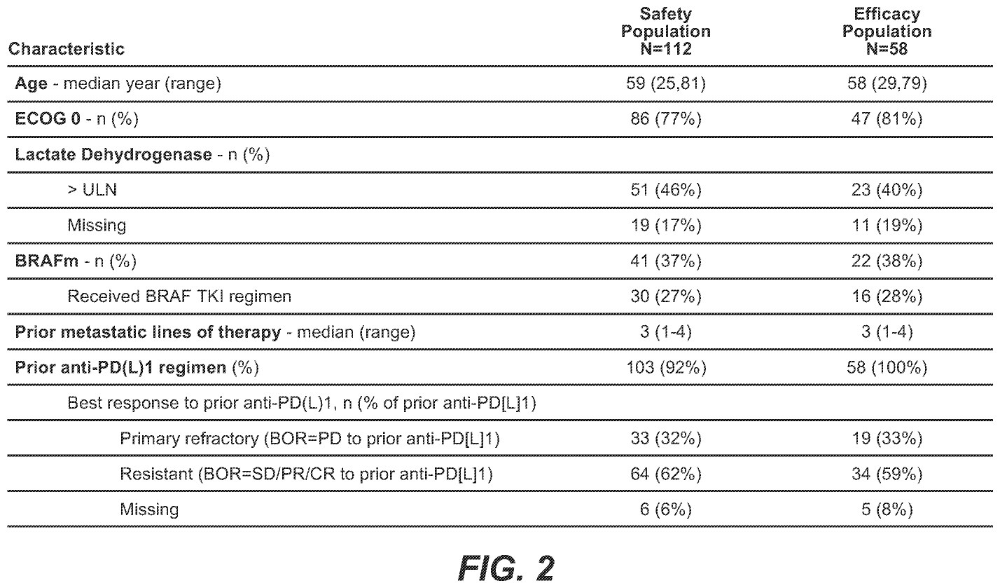

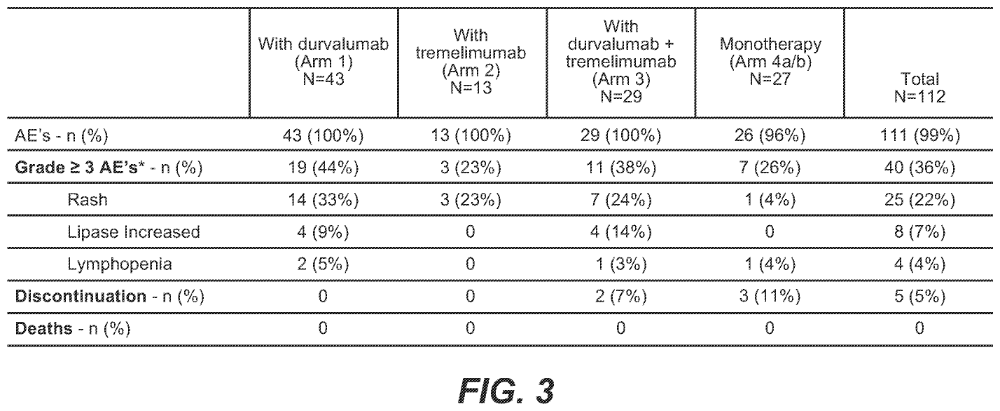

Maintenance period (applies to patients in Arms 1, 2 and 3): After the first 13 cycles, for patients who have achieved disease control, defined as SD or OR (PR or CR), the Maintenance period began at Cycle 14 (see FIG. 3-2 below). In Maintenance, patients received a reduced schedule of I tebentafusp (Day 15 of each cycle for an additional 12 cycles [Cycles 14-25] dosed with the durvalumab (Arms 1 and 3) or tremelimumab Q12W [Arm 2]). Dosing of the study medications during Maintenance continued according to the protocol-specified regimens until unacceptable toxicity, confirmed unequivocal disease progression per modified irRC, the patient completed Maintenance (Cycle 25), or a decision to discontinue participation by the treating physician or patient. Patients still with disease control (SD, PR or CR) at the end of Cycle 25 entered the Follow-up period.

The Arm 1, 2 and 3 Phase Ib DLT Observation period was the first 2 cycles of combination treatment (C1D1 through C2D28). The Arm 4 Phase Ib DLT Observation period will be from C1D22 to C2D14.

The Follow-up period consisted of the time from the last dose of study medication for a period of 90 days. Safety observations included reporting of all AEs and all SAEs in the same manner as the treatment period. Three safety observation visits were conducted in this time frame at days 30, 60, and 90 after the last dose of study medication due to the previous observations of late-onset toxicity associated with the checkpoint inhibitors (Wolchok, 2013; Larkin, 2015).

The Disease Progression Follow-up period was defined for all patients who discontinue treatment prior to PD per RECIST 1.1 but remained in the study. This group of patients was followed with imaging until evidence of PD per RECIST 1.1.

The Survival Follow-up period was initiate after either the 90-day Follow-up period (in patients who have discontinued for PD) or after the Disease Progression Follow-up period (for patients discontinuing study treatment prior to PD per RECIST 1.1) and continued until death. As possible, all patients were contacted by telephone and followed for survival until the end of the study is reached.

### 8.1.5. Study Treatment and Administration

For this study, the investigational drugs (or study medications) refer to tebentafusp, durvalumab, and tremelimumab. All study medications were supplied by the trial Sponsor, Immunocore.

In Arms 1, 2 and 3, tebentafusp was dosed weekly in Cycles 1-13 in the Treatment period. In all treatment arms, tebentafusp dosing at C1D1 and C1D8 could follow an intra-patient escalation to minimize toxicity. In an individual patient, the dose at C1D1 was capped at 20 mcg flat dose, and the dose at C1D8 was capped at 30 mcg (flat dose).

In treatment arm 1, 2 and 3, the dose administered at C1D15 and beyond was determined by the cohort level in Phase Ib arms and in the Phase II portion of the trial will be determined by the RP2D of each arm identified in the Phase Ib.

In treatment Arm 4, the dose of tebentafusp administered on C1D15 was 68 mcg; dose and schedule beyond C1D15 was determined by the cohort level/schedule in Phase Ib and in the Phase II portion of the trial will be determined by the RP2D/schedule identified in Phase Ib.

For patients in Arms 1, 2 or 3 with disease control (SD, PR or CR) at the completion of Cycles 1-13, a Maintenance period of a reduced schedule of tebentafusp was commenced (Cycles 14-25, tebentafusp dosed Q4W on Day 15 of every cycle); patients in Arm 4 will continued on their original schedule (see FIG. 1B).

All dosages prescribed and dispensed to patients and all dose changes during the study were recorded on the Dosage Administration Record electronic case report form (eCRF).

See Table 1 for Dose and Treatment Schedules.

TABLE 1
Dose and Treatment Schedules.

| | Pharmaceutical | | |
|---|---|---|---|
| | Form and | | |
| Study | Route of | | Frequency and/or |
| Treatments | Administration | Dose\hat{ } | Regimen |
| Tebentafusp | Concentrate | Arms 1-3: RP2D* | Arms 1-3: Every |
| | for solution | or cohort | week × 13 cycles, |
| | for infusion | assignment based | then every 4 |
| | (single use | on dose levels in | weeks(Day 15) |
| | vials) | Tables 6-2, 6-3, | Arm 4a: every |
| | | or 6-4. Dose at | week |
| | | C1D1 capped at | Arm 4b: between 2 |
| | | 20 mcg and dose | and 3 times per |
| | | at C1D8 capped | week from |
| | | at 30 mcg\hat{ }\hat{ } | C1D22 |
| | | Arm 4: RP2D* or | |
| | | cohort assignment | |
| | | based on dose | |
| | | levels in Table | |
| | | 6-5 and 6-6. | |
| | | 20 mcg (flat dose) | |
| | | on C1D1, 30 mcg | |
| | | (flat dose) C1D8 | |
| | | 68 mcg (flat dose) | |
| | | C1D15\hat{ }\hat{ } | |
| Durvalumab | Concentrate | Arm 1: RP2D* or | Every 4 weeks |
| | for Solution | cohort assignment | (Day 15) |
| | | in Phase Ib | |
| | for Infusion | Arm 3: RP2D* or | |
| | | cohort assignment | |
| | | in Phase Ib | |
| Tremelimumab | Concentrate | Arm 2: RP2D* or | Every 4 weeks |
| | for Solution | cohort assignment | (Day 15) for 7 |
| | for Infusion | in Phase Ib | doses, then every |
| | | Arm 3: RP2D* or | 12 weeks |
| | | cohort assignment | Every 4 weeks |
| | | in Phase Ib | (Day 15) for 4 |
| | | | doses then stop |

C#D# = Cycle # Day #; RP2D = recommended Phase II dose.
\hat{ }The RP2D for Arm 1 (durvalumab), Arm 2 (tremelimumab), and Arm 3 (durvalumab +
tremelimumab) with tebentafusp will be determined in Phase Ib.
*Starting dose levels for Phase Ib cohorts in Arms 1, 2, and 3 are as described herein.
\hat{ }\hat{ }Dosing of tebentafusp will follow an intra-patient escalation with the first 2 weekly doses capped. The initial doses at C1D1 and C1D8 are capped at 20 mcg (C1D1) and 30 mcg (C1D8). Arm 4 will also cap the C1D15 dose at 68 mcg.

For dose calculations of durvalumab and tremelimumab, the dose was calculated using the patient's Day 15 weight for each cycle. Dose adjustments for each dose were only needed for greater than 10% change in weight. Dosing day weight was used for dose calculations instead of weight at Day 15 of each cycle per institutional standard if alterations in scheduling were needed.

### 8.1.5.1 Tebentafusp

Tebentafusp was available in two formulations which have different concentrations as follows: 0.5 mg/mL and 0.2 mg/mL. The dose of tebentafusp delivered was the same regardless of which formulation is used. Each vial is designed for single use only and is not to be used to treat more than one patient for more than one dose.

Patients that begin treatment of either the 0.5 mg/mL or 0.2 mg/mL formulation remained on the same formulation until they are off active treatment.

Tebentafusp 0.5 mg/mL drug product was provided as a sterile, frozen solution in glass vials. Each glass vial will contain approximately 0.3 mL (extractable volume) with a concentration of 0.5 mg/mL. Tebentafusp 0.5 mg/mL drug product was stored below −60° C.

Tebentafusp 0.2 mg/mL drug product was provided as a sterile, refrigerated solution in glass vials. Each glass vial will contain approximately 0.5 mL (extractable volume) with a concentration of 0.2 mg/mL. Tebentafusp 0.2 mg/mL drug product was stored between 2° C. and 8° C.

Both formulations were supplied as concentrate for solution for infusion and require dilution prior to administration. However, the dose preparation method is different for each formulation and so caution MUST be exercised when dispensing and preparing doses. Detailed dose preparation instructions are provided in the study pharmacy manual and/or on the relevant pharmacy handling instructions. Once prepared for administration, the final infusion bag containing tebentafusp must be administered within 4 hours at room temperature from the time of initial puncture of the supplied drug vial.

The target duration of administration of tebentafusp was 15-20 minutes, per infusion. The entire content of the IV bag was infused using an infusion pump. The IV line was flushed after the contents of the IV bag are fully administered according to institutional policy to ensure the full dose is administered.

### 8.1.5.2 Durvalumab

Durvalumab was supplied as a 500 mg/vial solution for infusion after dilution. The solution contained 50 mg/mL durvalumab, 26 mM histidine/histidine hydrochloride, 275 mM trehalose dihydrate, and 0.02% (w/v) polysorbate 80; a pH of 6.0 and a density of 1.054 g/mL. The nominal fill volume was 10.0 mL. Durvalumab vials were stored at 2° C. to 8° C. (36° F. to 46° F.) and were not frozen. Drug product was kept in original packaging until use to prevent prolonged light exposure.

The dose of durvalumab for administration was prepared by the investigator's or site's designated Investigational Product Manager using aseptic technique. Total time from needle puncture of the durvalumab vial to the start of administration did not exceed: 24 hours at 2° C.-8° C. (36° F.-46° F.) and 4 hours at room temperature.

The volume of durvalumab (in mL) to add to the IV bag was calculated as follows: Dose (mL)=[patient weight (kg)×durvalumab dose level (mg/kg)] 50 mg/mL (where 50 mg/mL is durvalumab concentration). The corresponding volume of durvalumab was rounded to the nearest 10th mL (0.1 mL). Dose adjustments for each cycle were only needed for a greater than 10% change in weight. Weight was measured on Day 15 of each cycle for dose calculation; however, dosing day weight was used for dose calculations instead of weight at Day 15 of each cycle per institutional standard if alterations in scheduling were needed. Preparation of durvalumab and preparation of the IV bag were performed aseptically.

Durvalumab doses were administered using an IV bag containing 0.9% (w/v) saline or 5% (w/v) dextrose, with a final durvalumab concentration ranging from 1 to 15 mg/mL, and delivered through an IV administration set with a 0.2- or 0.22-μm filter.

Standard infusion time was 1 hour; however, if there are interruptions during infusion, the total allowed time should not exceed 8 hours at room temperature.

### 8.1.5.3 Tremelimumab

Tremelimumab was supplied as a 400 mg vial solution for infusion after dilution. The solution contained 20 mg/mL tremelimumab, 20 mM histidine/histidine-hydrochloride, 222 mM trehalose dihydrate, 0.27 mM disodium edetate dihydrate and 0.02% (w/v) polysorbate 80; a pH of 5.5 and a density of 1.034 g/mL. The nominal fill volume was 20 mL for the 400 mg vial. Tremelimumab vials are stored at 2° C. to 8° C. (36° F. to 46° F.) and were not frozen. Drug product was kept in original packaging until use to prevent prolonged light exposure.

The dose of tremelimumab for administration was prepared by the Investigator's or site's designated Investigational Product Manager using aseptic technique. Total time from needle puncture of the tremelimumab vial to the start of administration did not exceed: 24 hours at 2° C. to 8° C. (36° F. to 46° F.) and 4 hours at room temperature.

The dose was calculated using the following formula: Dose (mL)=[patient weight (kg)×tremelimumab dose level (mg/kg)] 20 mg/mL where 20 mg/mL is tremelimumab concentration. The corresponding volume of tremelimumab was rounded to the nearest 10th mL (0.1 mL). Dose adjustments for each cycle were only needed for a greater than 10% change in weight. Dosing day weight was used for dose calculations instead of weight at Day 15 of each cycle per institutional standard if alterations in scheduling was needed. Preparation of tremelimumab and preparation of the IV bag are to be performed aseptically.

Tremelimumab doses were administered using an IV bag containing 0.9% (w/v) saline or 5% (w/v) dextrose, with a final tremelimumab concentration ranging from 0.1 mg/mL to 10 mg/mL, and delivered through an IV administration set with a 0.2- or 0.22-μm filter.

Standard infusion time was 1 hour; however, if there were interruptions during infusion, the total allowed time should not exceed 8 hours at room temperature.

In Arm 2 (tremelimumab+tebentafusp), patients received tremelimumab at the assigned dose in Phase Ib and at the RP2D in Phase II Q4W (Day 15) for 7 doses and then Q12W. Patients who completed Cycle 13 with disease control entered the Maintenance period with reduced scheduling of tebentafusp (Q4W).

### 8.1.5.4 Durvalumab and Tremelimumab Administration

For durvalumab and tremelimumab combination therapy, tremelimumab was administered first; the durvalumab infusion started about 1 hour (maximum 2 hours) after the end of the tremelimumab infusion. If there were no clinically significant concerns after the first cycle, then, at the discretion of the Investigator, all other cycles of durvalumab were given immediately after the tremelimumab infusion had finished.

In Arm 3 (triplet), patients received the assigned dose of durvalumab+tremelimumab in Phase Ib or the RP2D of the combination with tebentafusp in Phase II. Tremelimumab dosing in Arm 3 was limited to Q4W (Day 15) for 4 doses and then stopped. Durvalumab dosing Q4W continued through the Treatment period (Cycles 1-13) and continue in Maintenance with durvalumab Q4W with a reduced schedule of tebentafusp.

### 8.1.6. Treatment Regimen

Patients enrolled in all 4 treatment arms of this study began treatment with single-agent tebentafusp for 2 doses on C1D1 and C1D8. In treatment Arm 1, 2 and 3, treatment with the checkpoint inhibitor (durvalumab and/or tremelimumab) in combination with tebentafusp began on C1D15 in Arms 1, 2, and 3. Durvalumab was administered on Day 15 of each cycle, in Arms 1 and 3 Q4W, until treatment discontinuation. Similarly, tremelimumab was administered on Day 15 of each cycle, Q4W for the first 7 doses in Arm 2, when dosing changed to Q12W. In Arm 3 (triplet), tremelimumab followed a reduced schedule of Q4W dosing on Day 15 of the first 4 cycles and then stopped. Tremelimumab did not resume in Arm 3 after the first 4 doses.

Patients enrolled in Arm 4 continued to receive only tebentafusp on C1D15, and then per the defined cohort dose and/or schedule on C1D22.

### 8.1.6.1 Infusion Timing and Order of Infusions

On a given infusion day in the clinic, in the doublet arms (Arms 1 and 2) durvalumab or tremelimumab were administered first (1 hour infusion), followed by a break of 60 minutes (up to 3 hours duration if clinically indicated) before administration of tebentafusp (15 to 20 minute infusion). In the triplet arm, tremelimumab was administered first; the durvalumab infusion was started approximately 1 hour (maximum 2 hours) after the end of the tremelimumab infusion, followed by a break of 60 minutes (up to 3 hours duration if clinically indicated) before administration of tebentafusp (15 to 20 minute infusion). If there were no clinically significant concerns after the first cycle, then, at the discretion of the investigator, all other cycles of durvalumab were given immediately after the tremelimumab infusion has finished, followed by a break of 60 minutes (up to 3 hours duration if clinically indicated) before administration of tebentafusp (15 to 20 minute infusion).

### 8.1.7. Outcome Measures

### 8.1.7.1 Primary Outcomes Measures

Phase 1b: Number of dose-limiting toxicities (DLT) were measured during the DLT observation period (i.e., about 12 months). DLT observation period for the Arms 1 to 3 Phase 1b cohorts was the first 2 cycles of treatment (C1D1 until C2D28). The DLT observation period for Arm 4 Phase Ib was from C1D22 to C2D14. A DLT was defined as an adverse event or abnormal laboratory value that occurs during the relevant DLT period which was assessed as having a suspected relationship to study drug, and unrelated to disease, disease progression, inter-current illness, or concomitant medications, occurred during the DLT Observation Period or is Grade 3 or higher per NCI CTCAE version 4.03, or as specified in the protocol.

Phase 2b: Aim of the Phase 2b study was to determine objective Response Rate (RECIST v1.1). Objective response rate was defined as the proportion of patients with a best response of CR or PR based on investigator assessment, as defined in RECIST v1.1. Phase 2b time frame was about 2 years.

### 8.1.7.2 Second Outcome Measures

Secondary outcome measures included:
1. Overall survival (Time Frame: 2 years): Time from the date of first dose until death due to any cause.
2. Safety: AEs and SAEs (Time Frame: 2 years): Safety incidence and severity of Adverse Events (AEs) and Serious Adverse Events (SAEs) including changes in laboratory. parameters, vital signs, and electrocardiograms (ECG).
3. Safety: Tolerability (Time Frame: 2 years): Dose interruptions
4. Safety: Tolerability (Time Frame: 2 years): Dose Reductions
5. Safety: Tolerability (Time Frame: 2 years): Dose Intensity
6. Serum Pharmacokinetics (Time Frame: 2 years): AUClast: Area under the curve (AUC) from time zero to the last measurable concentration sampling time (tlast) (mass×time×volume-1)
7. Serum Pharmacokinetics (Time Frame: 2 years): AUCinf: The AUC from time zero to infinity (mass×time×volume-1)
8. Serum Pharmacokinetics (Time Frame: 2 years): Cmax: Maximum Plasma Concentration
9. Serum Pharmacokinetics (Time Frame: 2 years): Tmax: The time to reach maximum (peak) plasma, blood, serum, or other body fluid drug concentration after single dose administration (time)
10. Serum Pharmacokinetics (Time Frame: 2 years): t1/2: Elimination half-life associated with the terminal slope (λz) of a semi logarithmic concentration-time curve (time)
11. Correlation of PD-L1 and gp100 (Time Frame: 2 years)
12. Progression Free Survival (Time Frame: 2 years): Correlation of gp100 and PD-L1 expression by immunohistochemistry evaluated in pre-treatment biopsies with anti-tumor activity.
13. Duration of Response (Time Frame: 2 years): Time from the date of first documented response until date of documented progression or death in the absence of disease progression. The median duration of response and corresponding 90% confidence interval will be presented.
14. Time to Response (Time Frame: 2 years): Time from initiation of therapy to the time that an OR per RECISTv1.1 is achieved.
15. Disease Control Rate (Time Frame: 2 years): Proportion of patients with either a best response of PR or CR or with SD over 24 weeks after first dose in the study. The DCR and associated 90% confidence interval will be presented by treatment arm.
16. Formation of Anti-drug Antibodies (Time Frame: 2 years): Incidence of anti-Tebentafusp, anti-durvalumab, and anti-tremelimumab antibody formation following multiple infusions of Tebentafusp alone and in combination with durvalumab and/or tremelimumab.

### 8.1.8. Eligibility Criteria

Inclusion Criteria:
1. Age≥18 years
2. Written informed consent was obtained from all patients prior to any study procedures
3. Patients with advanced non-uveal melanoma defined as unresectable stage III or metastatic stage IV disease. Patients with acral or mucosal melanoma were accepted. Patients with melanoma of unknown primary were accepted for Phase 1b escalation cohorts (Arms 1 to 5) but were excluded in Phase 2. NOTE: Patients with the diagnosis of UM were excluded from all cohorts.
4. Phase 1b (Arm 4 and Arm 5) and Phase 2: Patients with disease progression following initiation of treatment with an approved PD-(L)1 inhibitor. Patients with BRAF mutations were refractory to approved BRAF-inhibitor if clinically feasible. CTLA 4 inhibition therapy was accepted as a prior line of therapy or in combination with anti-PD-(L)1 therapy. For Phase 2, no prior chemotherapy in the advanced setting is permitted.
5. Phase 1b Arms 1-3: no restriction on prior therapy.
6. HLA-A*02:01 positive by central assay or by an 510K approved assay run in CLIA-certified laboratory.
7. Eastern Cooperative Oncology Group (ECOG) Performance Status of 0 or 1.
8. Life expectancy of at least 3 months.
9. Phase 2 cohorts only: Patients had measurable disease according to RECIST v1.1 criteria. Patients enrolled in Phase 1b cohorts had either measurable or only non-measurable disease (i.e., non-target lesion only).
10. Phase 1b (Arm 4) and Phase 2 cohorts only: Patients had a site of disease amenable to biopsy (i.e., must not also be a target lesion OR if a target lesion must be >2 cm), and were candidates for tumor biopsy according to the treating institution's guidelines. NOTE: Phase 1b Arms 1-3 and 5 patients were not required to have disease accessible to biopsy.
11. For Arms 1-3 only (i.e., applied only to patients assigned to receive tebentafusp in combination with checkpoint inhibitor[s]): Those receiving prior immunotherapy met all the following conditions:
   a. Must not have experienced an immune-related adverse event (irAE) where the irAE was the reason for permanent discontinuation of prior immunotherapy in the most recent prior treatment regimen.
   b. All irAEs while receiving prior immunotherapy must have resolved to ≤Grade 1 or baseline prior to screening for this study. Must not have experienced a ≥Grade 3 immune-related AE within the past 16 weeks or any Grade 4 life-threatening irAE (regardless of duration) or neurologic or ocular AE of any grade while receiving prior immunotherapy. (NOTE: Patients with endocrine AE of any grade were permitted to enroll if they were stably maintained on appropriate replacement therapy, but must not have had a history of adrenal crisis and be asymptomatic).
   c. Patients that were currently receiving chronic corticosteroid treatment (longer than 8 weeks duration) for management of pre-existing AEs, or patients with a history of chronic corticosteroid treatment longer than 8 weeks duration for AEs within 6 months of screening were excluded.

Exclusion Criteria
1. Presence of untreated or symptomatic central nervous system (CNS) metastases, leptomeningeal disease, or cord compression.
2. History of severe hypersensitivity reactions to study medications.
3. History of treatment-related interstitial lung disease/pneumonitis.
4. Impaired baseline organ function as evaluated by out-of-range laboratory values.
5. Clinically significant cardiac disease or impaired cardiac function.
6. Active autoimmune disease or a documented history of autoimmune disease.
7. Recent (<12 months of planned first dose of study treatment) active diverticulitis (Phase 1b combination arms).
8. Active infection requiring systemic antibiotic therapy. NOTE: Patients requiring systemic antibiotics for infection must have completed therapy before planned first dose of study treatment.
9. Known history of human immunodeficiency virus (HIV) infection. Testing for HIV status is not necessary unless clinically indicated or if required by local regulation.
10. Active hepatitis B virus (HBV) or hepatitis C virus (HCV) infection, currently requiring medical intervention, per institutional protocol. Testing for HBV or HCV status was not necessary unless clinically indicated or the patient had a history of HBV or HCV infection requiring treatment with an unknown status at time of enrollment. History of treated hepatitis was not exclusionary.
11. Malignant disease, other than that being treated in this study. Exceptions to this exclusion included the following: malignancies that were treated curatively and have not recurred within 2 years after completion of treatment; completely resected basal cell and squamous cell skin cancers; any malignancy considered to be indolent and that has never required therapy; and completely resected carcinoma in situ of any type.
12. Any medical condition that would have, in the investigator's judgment, prevented the patient's participation in the clinical study due to safety concerns, compliance with clinical study procedures or interpretation of study results.
13. Systemic anti-cancer therapy within 2 weeks of the planned first dose of study treatment. For cytotoxic agents that would have had major delayed toxicity and any prior immunotherapy approach, 3 weeks was indicated as washout period.
14. Presence of NCI CTCAE≥Grade 2 toxicity (except alopecia, peripheral neuropathy, and ototoxicity, which are excluded if ≥NCI CTCAE Grade 3) due to prior cancer therapy.
15. Systemic treatment with steroids or any other immunosuppressive drug use within 4 weeks of the planned first dose of study treatment, with the following exceptions:
   a. Treatment for well-controlled and asymptomatic adrenal insufficiency was permitted, but replacement dosing was limited to prednisone≤12 mg daily or the equivalent.
   b. Local steroid therapies (e.g., optic, ophthalmic, intra-articular, or inhaled medications) were acceptable.
   c. Premedication for allergy to contrast reagent or premedication regimen instituted per protocol.
   d. Steroids for management of CNS metastases>2 weeks prior to the planned first dose of study treatment.
16. Use of any live vaccines against infectious diseases within 4 weeks of initiation of study treatment.
17. Major surgery as defined by the investigator within 2 weeks of the first dose of study treatment.
18. Radiotherapy within 2 weeks of the first dose of study treatment, with the exception of palliative radiotherapy to a limited field, such as for the treatment of bone pain or a focally painful tumor mass.
19. Use of hematopoietic colony-stimulating growth factors (e.g., G-CSF, GMCSF, MCSF)≤2 weeks prior start or study treatment. NOTE: Patients must have completed therapy with hematopoietic colony-stimulating factors at least 2 weeks before the first dose of study treatment was given. An erythroid stimulating agent is allowed as long as it was initiated at least 2 weeks prior to the first dose of study treatment and the patient was not red blood cell transfusion dependent.
20. Pregnant or breast-feeding women.
21. Women of child-bearing potential who were sexually active with a non-sterilized male partner, unless they were using highly effective contraception during study treatment, and agreed to continue using such precautions for 1 week after the final dose of investigational product; cessation of birth control after this point was discussed with a responsible physician.
22. Male patients were surgically sterile or used double barrier contraception method and were not allowed to donate sperm from enrollment through treatment and for 3 months following administration of the last dose of study drug.
23. Patients who are relatives or dependents of the investigator.

### 8.1.9. Baseline Demographics for Study Participants

Baseline demographics for study participants are as shown in FIG. 2. Here, there was a distinction made between patients enrolled in the study whose cancer was refractory prior to anti-PD(L)1 therapy and patients whose cancer was resistant to prior anti-PD(L)1 therapy. In particular, 103 (92%) patients received prior anti-PD(L)1 therapy, of which 87% also received prior ipilimumab. Additionally, 38% were refractory to prior anti-PD(L)1 therapy (best prior response of progressive disease). Finally, 55% were resistant to prior anti-PD(L)1 therapy (best prior response of complete response, partial response or stable disease prior to progression.

### 8.1.10. Treatment-Related Adverse Events

A summary of tebentafusp-related adverse events for study Arms are shown in FIG. 3. Two DLTs occurred: prolonged grade 3 rash (Arm 1) and grade 2 diarrhea leading to treatment delay (Arm 2). Maximum tolerated dose was not reach in any cohort.

Related AEs that led to discontinuation included: Gr2/3 pancreatitis (Arm 3); Gr3 rash with Gr2 generalized edema (Arm 3); Gr2 epilepsy associated with new brain metastasis (Arm 4). AEs were generally consistent with the individual components of each Arm, and there were no treatment-related deaths. Overall, this suggests tebentafusp with anti-PD(L)1 and/or anti-CTLA4 in cutaneous melanoma (mCM) had an acceptable safety profile.

### 8.1.11. Results

8.1.11.1 Percent Change from Baseline in Tumor Size of Patients Who Received Prior Anti-P(L)1 Therapy, by Arm of Study

To assess how each study Arm effected tumor size, percent change from baseline in tumor size was measured for each Arm. Here, patients had previously received an anti-PD(L) therapy. Measurements were taken every 12 weeks after baseline.

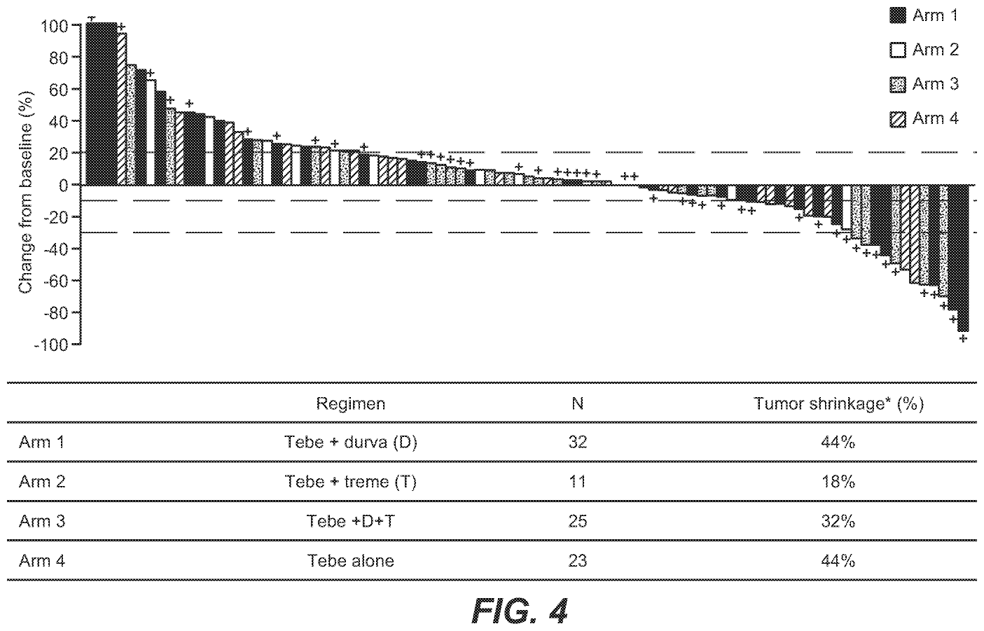

Results are shown in FIG. 4. Of the 91 patients, 6% had a confirmed RECIST partial response (PR). The median duration of response was 20 months (95% CI: 6-NR). Overall, 37% of the 91 patients had any tumor shrinkage as best change.

This data showed durable tumor shrinkage in 37% of prior anti-PD(L)1 mCM patients confirms tumor-killing activity of tebentafusp combined with durvalumab. Additionally, increase in OS was seen in patients who were resistant or refractory to prior anti-PD(L)1 therapy.

### 8.1.11.2 Durability of Tumor Shrinkage in Patients Who Received Prior Anti-PD(L)1 Therapy by Arm of Study

To assess how each study Arm effected tumor size, probability of tumor shrinkage was measured for each Arm over the course of the treatment periods. Here, patients had previously received an anti-PD(L)1 therapy.

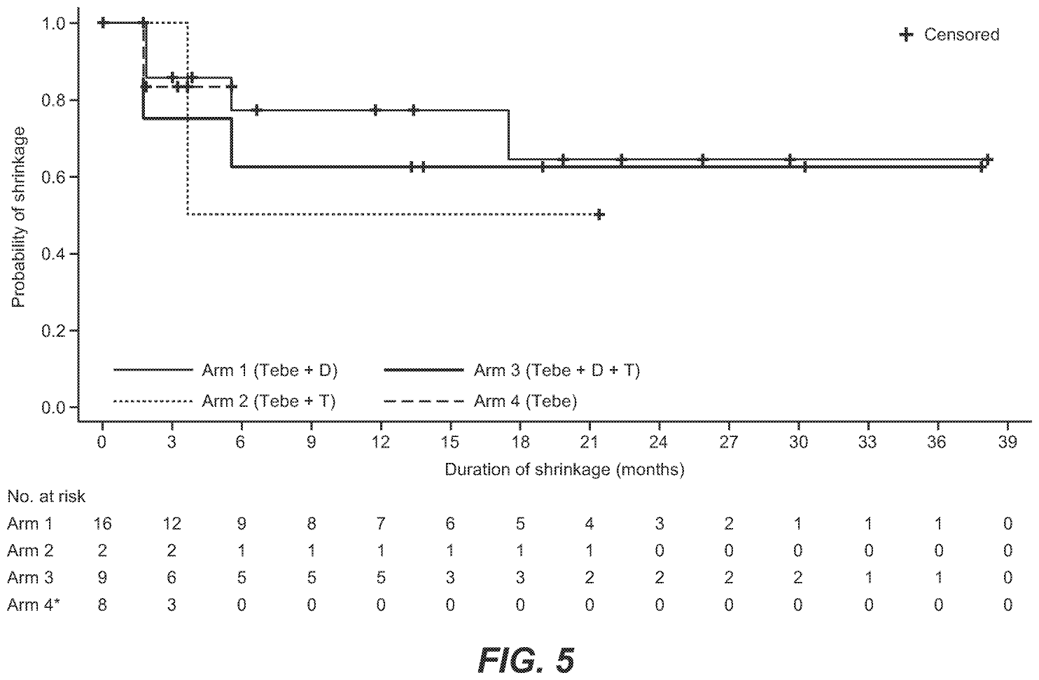

FIG. 5 shows that tumor shrinkage (as measured by probability of shrinkage versus duration of shrinkage) of target lesions (regardless of new lesions) was durable across study Arms. In particular, the probability of durable tumor shrinkage was high (>60%) even up to 2 years after initial administration. In fact, some patients had tumor shrinkage ongoing at 3+ years.

8.1.11.3 Percent Change from Baseline in Tumor Size, by Whether Patients were Refractory or Resistant to Prior Anti-PD(L)1 Therapy

Patients who were refractory to or relapsed following a prior checkpoint inhibitor treatment (i.e., anti-PD(L)1) were assessed following treatment according to the current study Arms.

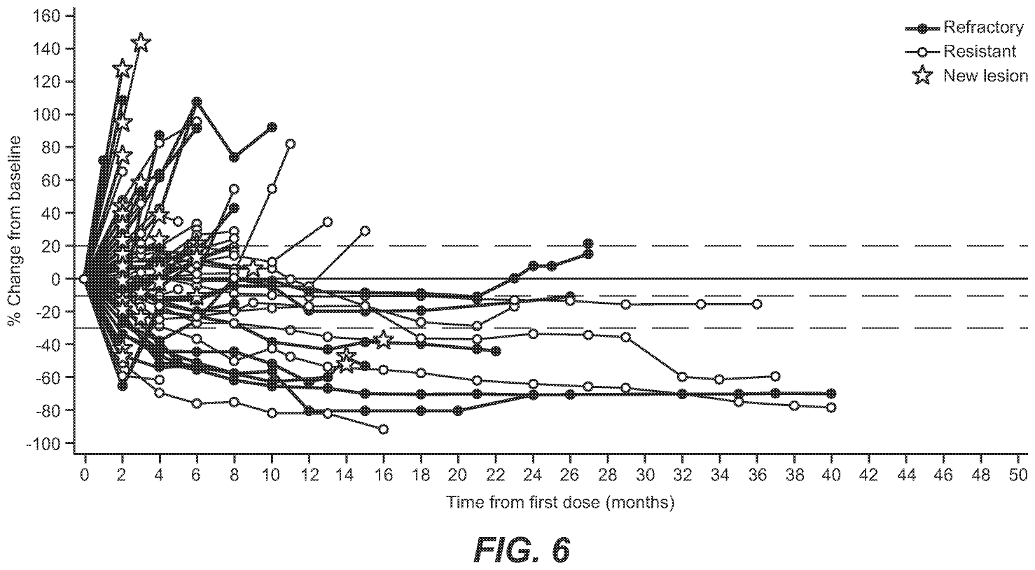

As shown in FIG. 6, sustained durable tumor shrinkage was seen in patients who were refractory or resistant to prior anti-PD(L)1. Some patients had tumor shrinkage that was durable despite appearance of new lesions.

8.1.11.4 OS by Whether Patients were Refractory or Resistant to Prior Anti-PD(L)1 Therapy

Overall survival was assessed for patients who were refractory to or relapsed following a prior checkpoint inhibitor treatment (i.e., anti-PD(L)1) and treated according to the current study Arms.

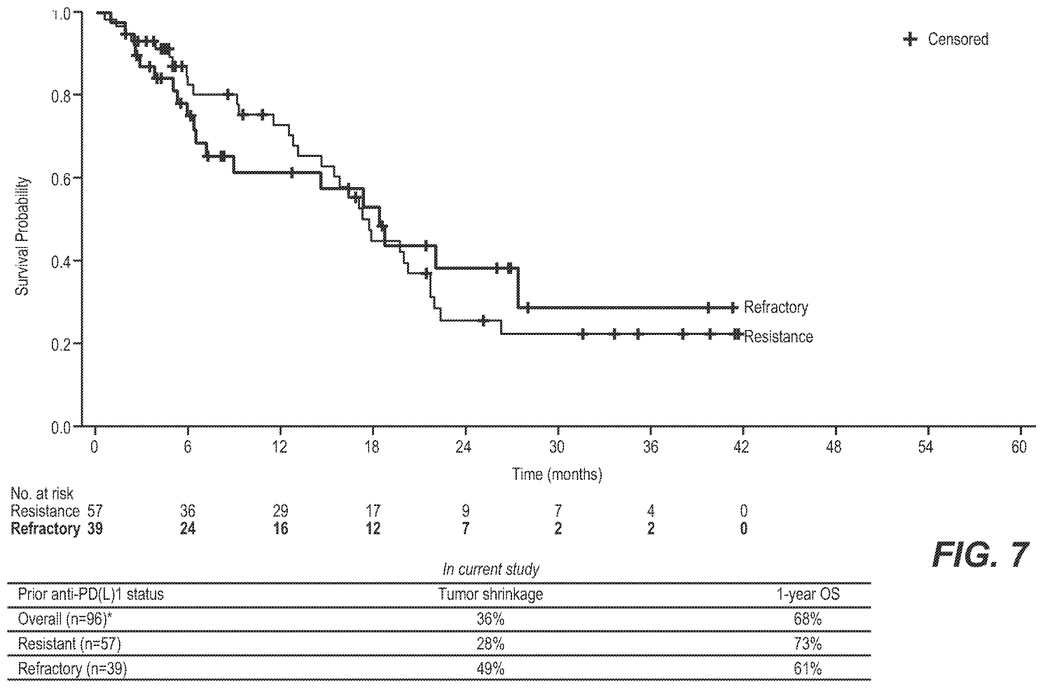

Results are shown in FIG. 7. Here, tumor shrinkage and an increase in OS was observed in patients regardless of whether they were resistant or refractory to prior anti-PD(L)1 therapy.

### 8.1.11.5 OS by Dose of Durvalumab in Patients Who Received Prior Anti-PD(L)1 Therapy

Focusing on Arm 1, overall survival was measured for patients who received prior anti-PD(L)1 therapy and treated according to Arm 1.

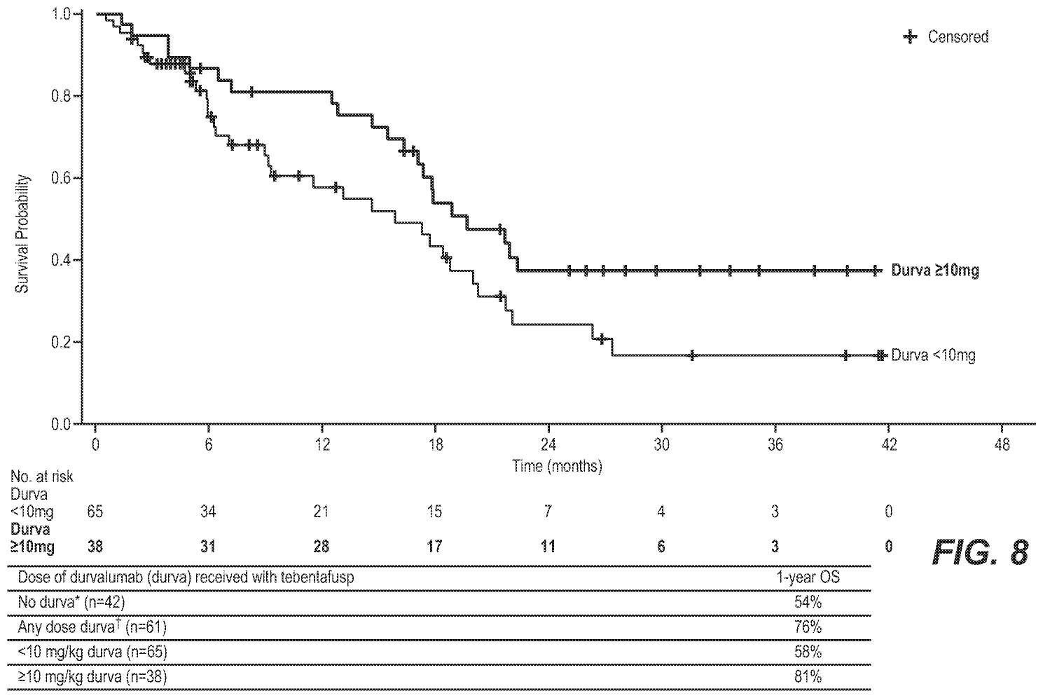

Results are shown in FIG. 8. With the caveat that the cohorts were not randomized, tebentafusp with any amount of durvalumab had a higher 1-yr OS (76%) compared to no durvalumab (54%). In particular, tebentafusp combined with ≥10 mg/kg durvalumab (a labelled dose of durvalumab) had the highest 1-year OS of 81%. This in contrast to recent investigational studies in anti-PD(L)1 resistant/refractory mCM have 1-yr OS of ˜50-60% (see, e.g., Arance A M et al., J. Clin. Oncol. 2021; 39 (suppl_15):9504-9504; Pereira C, et al., Ann. Oncol. 2020; 31(suppl_4):S672-S710; Zimmer L, et al., Eur. J. Cancer. 2017; 75:47-55; Weichenthal M, et al., J. Clin. Oncol. 2019; 37(suppl_15):9505-9505).

### 8.1.11.6 Association Between Tumor Shrinkage and Overall Survival for Tebentafusp and Durvalumab

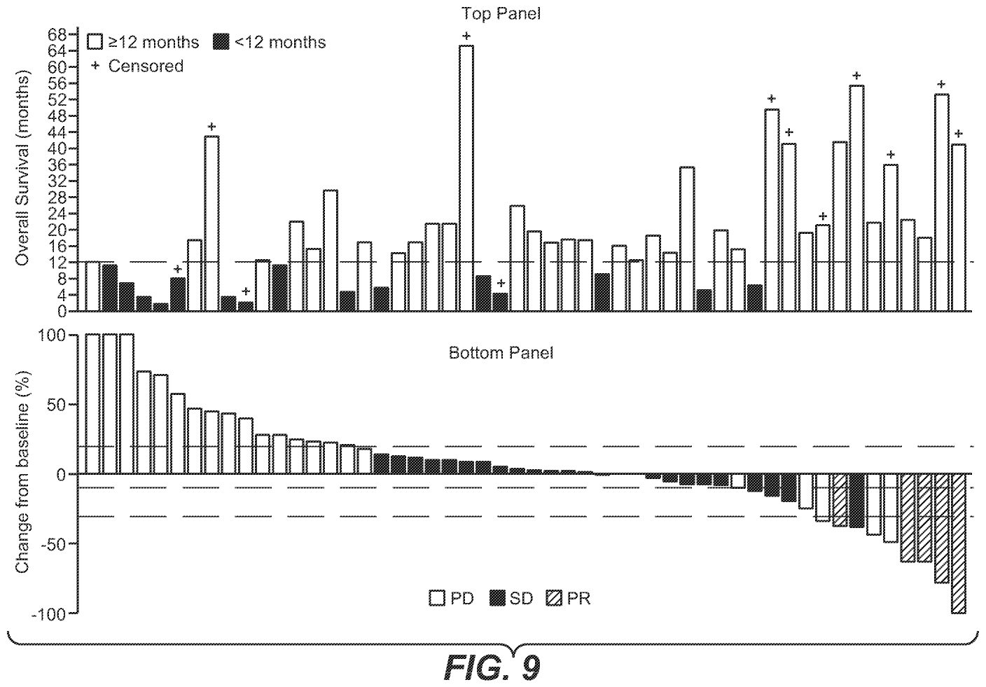

Next, Applicant assessed whether there was an association between tumor shrinkage and overall survival (OS) for patients in Arm 1 (tebentafusp and durvalumab). FIG. 9 top panel shows overall survival with treatment schedules having exceeded 12 months in purple and treatment schedules of less than or equal to 12 months in gray. FIG. 9 bottom panel shows change from baseline (%) for tumor measurements corresponding to the same patients immediately above in the top panel. Overall, the data show that there is a relationship between a negative change from baseline (%) with increased overall survival.

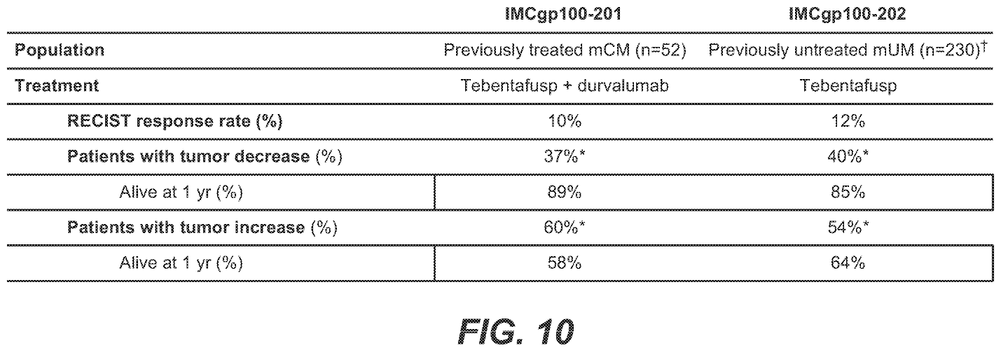

Applicant compared the readout from Arm 1 with previous data for tebentafusp treatment in metastatic uveal melanoma. As shown in FIG. 10, tumor shrinkage, and association with OS, comparable between mUM and mCM.

8.1.11.7 OS for Tebentafusp with Durvalumab or Durvalumab+Tremelimumab

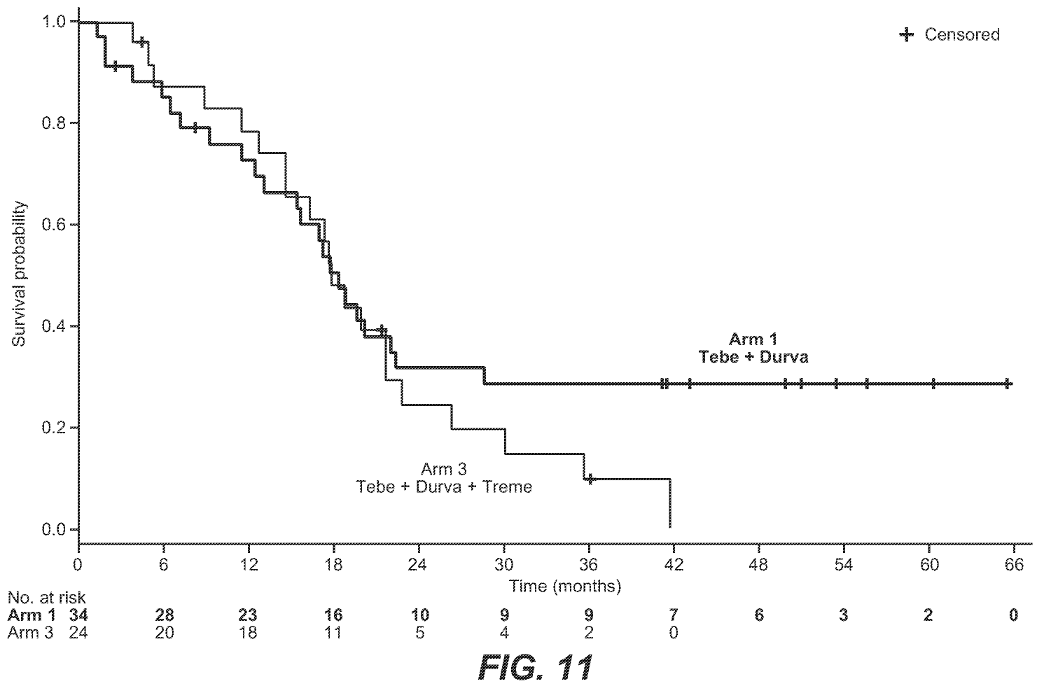

To determine whether adding tremelimumab to the combination of tebentafusp and durvalumab had the potential to increase overall survival, overall survival for patients in Arms 1 and 3 were compared. FIG. 11 and Table 2 shows that Arm 1 (tebentafusp with durvalumab) had a better survival probability than Arm 3 (tebentafusp with durvalumab and tremelimumab). Overall, this data suggest that including a CTLA-4 inhibitor in the treatment regimen is unlikely to improve overall survival.

TABLE 2

| Treatment Arm | 1-yr OS | 2-yr OS |
|---|---|---|
| Tebe + durva (Arm 1) | 73% | 32% |
| Tebe + durva + treme (Arm 3) | 78% | 24% |
| Benchmark¹ | 55% | N/A |

¹Arance A M et al. J Clin Oncol 2021; 39: S9504; Zimmer L et al. Eur J Cancer. 2017; 75: 47-55; Weichenthal M et al. J Clin Oncol 2019: 37: S15 9505-9505; Pires Da Silva I et al. J Clin Oncol 2020: 38: S15 10005-10005.

### 8.1.11.8 Conclusions

The above mentioned data supports the following conclusions. First, the data demonstrates a favorable safety profile for the combination of tebentafusp and an anti-PD(L)1 inhibitor, even with long term exposure. Second, patients receiving the combination therapy showed tumor shrinkage, and an association with OS, which was comparable between mUM and mCM. Third, treatment with tebentafusp and an anti-PD(L)1 inhibitor showed increased OS (1-yr˜75%) in heavily pre-treated mCM compared to benchmark (1-yr 55%). Overall, these data demonstrate a therapeutic benefit for metastatic cutaneous melanoma patients treated with the combination of tebentafusp and an anti-PD(L)1 inhibitor.

### 9. EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A method of treating melanoma in a patient, comprising:
administering to a patient who has or is suspected of having melanoma:
a therapeutically effective amount of tebentafusp; and
a therapeutically effective amount of a first immune checkpoint inhibitor.

2. A method of treating melanoma in a patient, comprising:
administering to a patient who is refractory to or relapsed following a prior checkpoint inhibitor treatment:
a therapeutically effective amount of tebentafusp; and
a therapeutically effective amount of a first immune checkpoint inhibitor.

3. The method of claim 2, wherein the prior immune checkpoint inhibitor treatment comprises an inhibitor of PD(L)1, CTLA-4, or a combination thereof.

4. The method of any one of claims 1-3, further comprising administering a second immune checkpoint inhibitor.

5. The method of any one of claims 1-4, wherein the first immune checkpoint inhibitor or the second immune checkpoint inhibitor is an inhibitor of PD(L)1.

6. The method of claim 5, wherein the inhibitor is a monoclonal antibody that binds PD(L)1.

7. The method of claim 6, wherein the monoclonal antibody that binds PD(L)1 is selected from:
durvalumab, atezolizumab, BMS-936559, avelumab, pembrolizumab, nivolumab, dostarlimab, and cemiplimap.

8. The method of claim 7, wherein the monoclonal antibody that bind PD(L)1 is durvalumab.

9. The method of any one of claims 1-8, wherein the first immune checkpoint inhibitor and/or the second immune checkpoint inhibitor is an inhibitor of CTLA-4.

10. The method of claim 9, wherein the inhibitor is a monoclonal antibody that binds CTLA-4.

11. The method of claim 10, wherein the monoclonal antibody that binds CTLA-4 is selected from: tremelimumab, ipilimumab, quavonlimab, zalifrelimab, GIGA-564, CBT-509, and AGEN1181.

12. The method of claim 11, wherein the monoclonal antibody that binds CTLA-4 is tremelimumab.

13. The method of any one of claims 1-12, wherein the melanoma is cutaneous melanoma.

14. The method of any one of claims 1-13, wherein the cutaneous melanoma is metastatic cutaneous melanoma.

15. The method of any one of claims 1-14, wherein the melanoma is refractory or resistant.

16. The method of claim 15, wherein the patient was previously treated with an inhibitor of PD(L)1.

17. The method of any one of claims 1-16, wherein the patient has a BRAF mutation.

18. The method of claim 17, wherein the patient was previously treated with an approved BRAF-based therapy.

19. The method of any one of claims 1-18, wherein the patient is HLA-A2-positive.

20. The method of any one of claims 1-19, wherein the tebentafusp and the first immune checkpoint inhibitor are administered separately.

21. The method of any one of claims 1-20, wherein the tebentafusp is administered weekly.

22. The method of any one of claims 1-21, wherein the first immune checkpoint inhibitor is administered once every four weeks.

23. The method of any one of claims 4-22, wherein the second immune checkpoint inhibitor is administered once every four weeks.

24. The method of any one of claims 4-23, wherein the first immune checkpoint inhibitor and the second immune checkpoint inhibitor are administered concurrently.

25. The method of any one of claims 4-23, wherein the first immune checkpoint inhibitor and the second immune checkpoint inhibitor are administered separately.

26. The method of any one of claims 1-25, wherein the first immune checkpoint inhibitor, the second immune checkpoint inhibitor, or both, are first administered about 15 days after the first administration of the tebentafusp.

27. The method of any one of claims 1-26, wherein the tebentafusp is administered at a dose ranging from about 10 mcg to about 68 mcg.

28. The method of any one of claims 1-26, wherein the tebentafusp is administered at a dose ranging from about 10 mcg to about 50 mcg.

29. The method of any one of claims 1-26, wherein the tebentafusp is administered at a dose ranging from about 68 mcg to about 200 mcg.

30. The method of any one of claims 1-29, wherein the first immune checkpoint inhibitor, the second immune checkpoint inhibitor, or both, are administered at a dose ranging from about 1 mg/kg to about 20 mg/kg.

31. The method of any one of claims 1-29, wherein the first immune checkpoint inhibitor, the second immune checkpoint inhibitor, or both, are administered at a dose ranging from about 0.5 mg/kg to about 10 mg/kg.

32. The method of any one of claims 1-29, wherein the first immune checkpoint inhibitor, the second immune checkpoint inhibitor, or both, are administered at a dose ranging from about 0.5 mg/kg to about 1 mg/kg.

33. The method of any one of claims 1-18, wherein the first immune checkpoint inhibitor is durvalumab.

34. The method of claim 33, wherein the tebentafusp is administered at a dose ranging from about 10 mcg to about 68 mcg and the durvalumab is administered at a dose ranging from about 1 mg/kg to about 20 mg/kg.

35. The method of claims 33 or 34, wherein the tebentafusp is administered about once weekly.

36. The method of any one of claims 33-35, wherein the durvalumab is administered about once every four weeks.

37. The method of claim 36, wherein the durvalumab is first administered about 15 days after the first administration of the tebentafusp.

38. The method of any one of claims 1-18, wherein the first immune checkpoint inhibitor is tremelimumab.

39. The method of claim 38, wherein the tebentafusp is administered at a dose ranging from about 10 mcg to about 50 mcg and the tremelimumab is administered at a dose ranging from about 0.5 mg/kg to about 10 mg/kg.

40. The method of claims 38 or 39, wherein the tebentafusp is administered about once weekly.

41. The method of any one of claims 38-40, wherein the tremelimumab is administered about once every four weeks.

42. The method of claim 41, wherein the tremelimumab is first administered about 15 days after the first administration of the tebentafusp.

43. The method of any one of claims 4-18, wherein the first immune checkpoint inhibitor is durvalumab and the second immune checkpoint is tremelimumab.

44. The method of claim 43, wherein the tebentafusp is administered at a dose ranging from about 10 mcg to about 50 mcg, the durvalumab is administered at a dose ranging from about 1 mg/kg to about 20 mg/kg, and the tremelimumab is administered at a dose ranging from about 0.5 mg/kg to about 1 mg/kg.

45. The method of claim 43 or 44, wherein the tebentafusp is administered about once weekly.

46. The method of any one of claims 43-45, wherein the durvalumab is administered about once every four weeks.

47. The method of claim 46, wherein the durvalumab is first administered about 15 days after the first administration of the tebentafusp.

48. The method of any one of claims 43-47, wherein the tremelimumab is administered about once every four weeks.

49. The method of claim 48, wherein the tremelimumab is first administered about 15 days after the first administration of the tebentafusp.

50. The method of any one of claims 43-49, wherein the first immune checkpoint inhibitor and the second immune checkpoint inhibitor are administered concurrently.

51. The method of any one of claims 43-49, wherein the first immune checkpoint inhibitor and the second immune checkpoint inhibitor are administered separately.

52. The method of any one of claims 1-51, wherein tebentafusp is administered via intravenous infusion, subcutaneous infusion, intramuscular infusion, enteral administration, inhalation, or intranasal.

53. The method of any one of claims 1-52, wherein the first immune checkpoint inhibitor, the second immune checkpoint inhibitor, or both are administered via intravenous infusion, subcutaneous infusion, intramuscular infusion, enteral administration, inhalation, or intranasal.

54. A method of treating metastatic cutaneous melanoma in a patient, comprising:
administering:
tebentafusp at a dose ranging from about 10 mcg to about 68 mcg; and
a PD(L)1 inhibitor at a therapeutically effective amount,
wherein the tebentafusp is administered about once weekly,
wherein the PD(L)1 inhibitor is administered about once every four weeks, and
wherein the PD(L)1 inhibitor is first administered about 15 days after the first administration of the tebentafusp.

55. The method of claim 54, wherein the PD(L)1 inhibitor is a monoclonal antibody that binds PD(L)1.

56. The method of claim 55, wherein the monoclonal antibody that binds PD(L)1 is selected from: durvalumab, atezolizumab, BMS-936559, and Avelumab.

57. The method of claim 56, wherein the PD(L)1 inhibitor is durvalumab.

58. A method of treating metastatic cutaneous melanoma in a patient, comprising:
administering:
tebentafusp at a dose ranging from about 10 mcg to about 68 mcg; and
durvalumab at a dose ranging from about 1 mg/kg to about 20 mg/kg,
wherein the tebentafusp is administered about once weekly,
wherein the durvalumab is administered about once every four weeks, and
wherein the durvalumab is first administered about 15 days after the first administration of the tebentafusp.

59. A method of treating metastatic cutaneous melanoma in a patient, comprising:
administering:
tebentafusp at a dose ranging from about 10 mcg to about 50 mcg; and
a CTLA-4 inhibitor at a therapeutically effective amount,
wherein the tebentafusp is administered about once weekly,
wherein the CTLA-4 inhibitor is administered about once every four weeks, and
wherein the CTLA-4 inhibitor is first administered about 15 days after the first administration of the tebentafusp.

60. The method of claim 59, wherein the CTLA-4 inhibitor is a monoclonal antibody that binds CTLA-4.

61. The method of claim 60, wherein the monoclonal antibody that binds CTLA-4 is selected from: tremelimumab, ipilimumab, quavonlimab, zalifrelimab, GIGA-564, CBT-509, and AGEN1181.

62. The method of claim 61, wherein the CTLA-4 inhibitor is tremelimumab.

63. A method of treating metastatic cutaneous melanoma in a patient, comprising:
administering:
tebentafusp at a dose ranging from about 10 mcg to about 50 mcg; and
tremelimumab at a dose ranging from about 0.5 mg/kg to about 10 mg/kg,
wherein the tebentafusp is administered about once weekly,
wherein the tremelimumab is administered about once every four weeks, and
wherein the tremelimumab is first administered about 15 days after the first administration of the tebentafusp.

64. A method of treating metastatic cutaneous melanoma in a patient, comprising:
administering:
tebentafusp at a dose ranging from about 10 mcg to about 50 mcg;
a PD(L)1 inhibitor at a therapeutically effective amount; and
a CTLA-4 inhibitor at a therapeutically effective amount,
wherein the tebentafusp is administered about once weekly,
wherein the PD(L)1 inhibitor is administered about once every four weeks,
wherein the CTLA-4 inhibitor is administered about once every four weeks, and
wherein the PD(L)1 inhibitor and the CTLA-4 inhibitor are first administered about 15 days after the first administration of the tebentafusp.

65. The method of claim 64, wherein the inhibitor is a monoclonal antibody that binds PD(L)1.

66. The method of claim 65, wherein the monoclonal antibody that binds PD(L)1 is selected from: durvalumab, atezolizumab, BMS-936559, and Avelumab.

67. The method of claim 66, wherein the PD(L)1 inhibitor is durvalumab.

68. The method of any one of claims 64-67, wherein the inhibitor is a monoclonal antibody that binds CTLA-4.

69. The method of claim 68, wherein the monoclonal antibody that binds CTLA-4 is selected from: tremelimumab, ipilimumab, quavonlimab, zalifrelimab, GIGA-564, CBT-509, and AGEN1181.

70. The method of claim 69, wherein the CTLA-4 inhibitor is tremelimumab.

71. A method of treating metastatic cutaneous melanoma in a patient, comprising:
administering:
tebentafusp at a dose ranging from about 10 mcg to about 68 mcg;
durvalumab at a dose ranging from about 1 mg/kg to about 20 mg/kg; and
tremelimumab at a dose ranging from about 0.5 mg/kg to about 10 mg/kg,
wherein the tebentafusp is administered about once weekly,
wherein the durvalumab is administered about once every four weeks,
wherein the tremelimumab is administered about once every four weeks, and
wherein the durvalumab and the tremelimumab are first administered about 15 days after the first administration of the tebentafusp.
